(19) United States
(12) Patent Application Publication
(10) Pub. No.: US 2016/0345952 A1
(43) Pub. Date: Dec. 1, 2016

(54) MINIMALLY INVASIVE DEVICES, SYSTEMS AND METHODS FOR TREATING THE SPINE

(71) Applicant: Spinal USA, Inc., Parsippany, NJ (US)

(72) Inventors: Donald Kucharzyk, Crete, IL (US); Stephen Termyna, Boonton, NJ (US); Thomas W. Winegar, Hawthorne, NJ (US); Luis A. Arellano, Fair Lawn, NJ (US); John Wilson, Brandon, MS (US); Melissa D. Coale, Chester, NJ (US); Rosemary M. Garofalo, Hamden, CT (US)

(21) Appl. No.: 15/115,140

(22) PCT Filed: Jan. 28, 2015
(86) PCT No.: PCT/US15/13188

§ 371 Date: Jul. 28, 2016

Related U.S. Application Data
(60) Provisional application No. 61/933,272, filed on Jan. 29, 2014.
(60) Provisional application No. 62/046,635, filed on Sep. 5, 2014.

(51) Int. Cl. A61B 17/02; A61B 90/00; A61B 17/00; A61B 17/70

(52) U.S. Cl. CPC A61B 17/0206; A61B 17/7037; A61B 17/7077; A61B 17/708; A61B 17/7082; A61B 90/03; A61B 17/00234; A61B 2090/037

(57) ABSTRACT

Devices and methods are provided for surgical retraction with a minimally invasive, maximum access surgical system. The surgical system can include anchor extensions that can be attached to bone screws. The bone screws can be inserted into a pedicle of a vertebral body. A retractor can be attached to anchor extensions connected to adjacent vertebrae on an operational side, and the retractor can be attached to anchor extensions connected to adjacent vertebrae on a contralateral side. The retractor can be used to distract the vertebral disc space between the adjacent vertebrae.

### RELATED APPLICATIONS

This application claims the priority benefit of U.S. Provisional Application Nos. 61/933,272, filed Jan. 29, 2014, and 62/046,635, filed Sep. 5, 2014, each of which is hereby incorporated by reference herein in its entirety. Additional features and aspects of some advantageous systems and methods that may be utilized in combination with embodiments found in this application are described in U.S. patent applications incorporated by reference herein, including, for example, U.S. Provisional Application No. 61/676,856, filed Jul. 27, 2012, and U.S. patent application Ser. No. 13/952,324, filed Jul. 26, 2013, published as US 2014/0031874 A1, which are expressly incorporated by reference herein in their entireties so as to form part of this specification.

### BACKGROUND OF THE DISCLOSURE

Field of the Disclosure

The present application relates to devices, systems and methods for treating the spine. In certain embodiments, the present application relates to devices, systems and methods for providing spinal stabilization, such as a spinal fusion. In particular, certain embodiments relate to minimally invasive devices and methods for delivering fixation devices and implants into the spine.

Description of the Related Art

Spinal bone and disc degeneration can occur due to trauma, disease or aging. Such degeneration can cause abnormal positioning and motion of the vertebrae, which can subject nerves that pass between vertebral bodies to pressure, thereby causing pain and possible nerve damage to a patient. In order to alleviate the pain caused by bone degeneration, it is often helpful to maintain the natural spacing between vertebrae to reduce the pressure applied to nerves that pass between vertebral bodies.

To maintain the natural spacing between vertebrae, spinal stabilization devices are often provided to promote spinal stability. These spinal stabilization devices can include fixation devices, such as spinal screws, which are implanted into vertebral bone. The fixation devices work in conjunction with other implanted members, such as rod members, to form stabilization systems.

Spinal stabilization devices are often used in conjunction with spinal fusion techniques, which can increase stability of the spacing between vertebrae by fusing adjacent vertebrae together. Two of the most common spinal fusion techniques are the transforaminal lumbar interbody fusion (TLIF) and the posterior lumbar interbody fusion (PLIF).

Conventional stabilization systems and techniques often require open surgeries and other invasive procedures in order to deliver the implants into the body. These invasive procedures often cause a great deal of pain and trauma to the patient, and require a substantial recovery time. Minimally invasive (MIS) and maximal access (MAS) systems and methods exist, but they are relatively new and with room for improvement.

### SUMMARY OF THE DISCLOSURE

Various embodiments described herein relate to a minimally invasive retractor access system that can be used as part of a minimally disruptive muscle sparing approach to the spine. Embodiments described herein can be used for single and multi-level spinal fusions. Embodiments described herein can also be used for bilateral distraction of a spinal disc space during a spinal procedure, such as by distracting from devices attached to bone screws on both sides of a spine. Among other benefits, this can help when distracting tight disc spaces.

In some embodiments, a minimally invasive surgical system for treating the spine can include at least one blade assembly. The blade assembly can have a blade with a central section having a central bore and wings extending outward from the central section on opposite sides thereof. The wings also extend to a location below a bottom of the central section, forming a gap bounded by the wings on two sides and the bottom of the central section on a third side. The blade assembly can also have a post extending from an upper surface of the blade and a shaft positioned at least partially within the central bore of the blade, the shaft having an externally threaded lower section and a central shaft bore that runs through the length of the shaft. The shaft can be rotatable within the central bore of the blade.

In some embodiments, the blade assembly can include an attachment section extending from the blade. In some embodiments, the shaft can have a plurality of vertical grooves extending around at least a portion of the shaft. In some embodiments, the blade assembly can further include a locking pin positioned within the attachment section, the locking pin moveable between a locked position in which a point on one end of the locking pin engages at least one of the vertical grooves of the blade shaft, substantially preventing rotation of the shaft, and an unlocked position in which the point is disengaged from the vertical grooves. In some embodiments, the locking pin can also be biased toward the locked position. In some embodiments, the vertical grooves are angled relative to a diameter of the shaft bore such that the locking pin in the locked position only prevents rotation of the shaft in one direction.

In some embodiments, the externally threaded lower section extends at least partially into the gap. In some embodiments, the surgical system can further include a pedicle screw having a screw shank and a housing attached to an upper end of the screw shank. The housing can have internal threading configured to receive the external threads of the lower section of the blade shaft.

In some embodiments, the surgical system can further include a retractor having a cross bar with a plurality of arms extending from the cross bar. In some embodiments, at least one of the arms can move relative to the cross bar to thereby change a distance between the arms. In some embodiments, each arm at its end farthest from the cross bar can have a collar configured to attach to the post of the blade assembly. In some embodiments, an attachment assembly can be connected to one of the arms, the attachment assembly configured to attach to a minimally invasive tower access device.

In some embodiments, the retractor can have a first set of arms extending from the cross bar on a first side of the cross bar, and a second set of arms extending from the cross bar on a second side of the cross bar. Each arm of the first set can have at least one articulating section. The arms of the first set can be moved closer together or further apart and the arms of the second set can be moved closer together or further apart. In some embodiments, each arm of the second set can be configured to attach to a minimally invasive tower access device. In some embodiments, when the arms of the first set are attached to blade assemblies that are attached to pedicle screws positioned within pedicles on the first side of a pair of adjacent vertebrae, and when the arms of the second set are attached to minimally invasive tower access devices that are attached to pedicle screws positioned within pedicles on the second side of the pair of adjacent vertebrae, the arms of each set can be simultaneously moved further apart to distract the disc space between the pair of adjacent vertebrae.

In some embodiments, a medial blade assembly can be configured to attach to at least one of the arms, the medial blade assembly comprising a medial blade configured to be movable toward the cross bar. In some embodiments, the medial blade assembly is movable in a direction toward at least one of the arms.

In some embodiments, a minimally invasive surgical system for treating the spine includes a plurality of pedicle screws for insertion into the pedicles of adjacent vertebrae, with each of the pedicle screws having an upper portion that is internally threaded. The system can also include a plurality of retractor blades, each of the blades having an externally threaded lower section configured to engage the internally threaded upper portion of a corresponding pedicle screw. The system can further include a plurality of retractor arms configured to move the retractor blades closer together or farther apart. In some embodiments, the system can further include a plurality of minimally invasive tower access devices configured to engage at least some of the plurality of pedicle screws. In some embodiments, the plurality of retractor arms includes at least some retractor arms configured to engage the plurality of minimally invasive tower access devices to move the minimally invasive tower access devices closer together or farther apart. In some embodiments, the system can further include an implant configured to be delivered between the plurality of retractor blades or between the plurality of tower access devices.

In various embodiments, a minimally invasive surgical system for treating the spine can include multiple pedicle screws, each pedicle screw configured to be positioned in a respective pedicle of at least a first vertebra and a second vertebra adjacent the first vertebrae. The system can also include multiple pedicle screw extensions, each extension configured to engage a respective pedicle screw. The system can further include a retractor body having a cross bar, a plurality of arms extending from the cross bar with at least two of the arms movable relative to each other, and four connection assemblies, each connection assembly attached to an arm and configured to attach to a respective pedicle screw extension and pedicle screw. In some embodiments, moving the at least two arms apart from each other moves the connection assemblies attached to pedicle screws positioned in the pedicles of the first vertebra apart from the connection assemblies attached to pedicle screws positioned in the pedicles of the second vertebra.

In some embodiments, a retractor system can be sized and configured to enable a multi-level surgery. For example, some embodiments can include six screws and connectors. Accordingly, in some embodiments you can distract between the first two pairs of screws (e.g., a left pair and the middle pair). Once the interbody is placed, the retractor can be moved to the second two pairs of screws (e.g., the middle pair and right pair) in order to distract the next level. In some embodiments, the retractor is long enough to distract directly between the left pair and the right pair.

In some embodiments, the retractor body can include two arms and each arm can be attached to at least one connection assembly. In some embodiments, at least one of the pedicle screw extensions can be or attach to blade assemblies and at least one of the pedicle screw extensions can be or attach to tower access devices. In some embodiments, the plurality of arms can extend from the cross bar on the same side of the cross bar.

In various embodiments, a screwdriver and screw extender can be used to help attach screws used in spinal procedures. The screwdriver can have a section with ridges, and the screw extender can have a moveable locking plate with an edge or tooth configured to releasably engage the ridged section of the screwdriver. In some embodiments, the locking plate can be biased into engagement with the screwdriver. In some embodiments, a user can manually release the locking plate from engagement with the screwdriver. In some embodiments, a screw extender can have multiple locking plates or other components configured to releasably engage the screwdriver.

In some embodiments, a minimally invasive surgical system for treating the spine comprises at least one anchor extension attachment assembly. The anchor extension assembly comprises a tube having a central section with a central bore. An arm attachment portion is coupled to the tube and configured to be coupled to a retractor arm. First and second flange portions are coupled to a spring-loaded hinge portion that is coupled to the tube, the second flange portion has a hook portion configured to releasably engage a portion of the retractor arm.

In some embodiments, a minimally invasive surgical system for treating the spine comprises at least one medial blade assembly. The medial blade assembly comprises an assembly attachment portion configured to be coupled with a retractor arm. A medial blade positioning portion is configured to be adjustably coupled to the assembly attachment portion. A medial blade arm is configured to be adjustably coupled to the medial blade positioning portion and configured to be coupled to a medial blade.

In some embodiments, a minimally invasive surgical system for treating the spine comprises at least one medial blade. The medial blade comprises a blade body having a proximal portion and a distal portion wherein the proximal portion is thicker than the distal portion. A slot is defined along a length of the blade body and configured to receive a lighting element tool. A proximal portion of the blade body comprises an opening and is configured to receive a top-loading self-retaining attachment feature to secure the blade to a top surface of a retractor tool support portion of a surgical retractor system.

In some embodiments, a minimally invasive surgical system for treating the spine comprises at least one spacing tool. The spacing tool comprises first and second handle portions. A ratchet mechanism is coupled to the first and second handle portions and configured to hold the tool in an expanded position in a first open configuration. A pivot portion is coupled to the first and second handle portions. First and second arms extend distally from the first and second handle portions. First and second spacer portions coupled to distal portions of the first and second arms have substantially hemi-spherical cross-sectional portions. The tool is configured to be actuated from a closed configuration to an open configuration and held in the open configuration by the ratchet mechanism to provide a working space at a surgical location.

In some embodiments, a minimally invasive surgical system for treating the spine comprises a plurality of anchor extension attachment assemblies. Each of the plurality of anchor extension attachment assemblies comprises a shaft having a central section with a central bore. An arm attachment portion is configured to be coupled to a retractor arm. A spring-loaded locking clip is coupled to the arm attachment portion and configured to releasably engage the retractor arm. The shaft is configured to be coupled to anchor extension assemblies comprising extended tab percutaneous screws.

Methods are also described herein. In some embodiments, a minimally invasive surgical method for treating the spine includes delivering a first pedicle screw into the pedicle of a first vertebra and a second pedicle screw into the pedicle of a second vertebra, the first and second pedicle screws having threaded upper portions. The method can also include distracting a disc space between the first and second vertebra by moving retractor blades engaged with the threaded upper portions of the first and second pedicle screws, and delivering an implant between the retractor blades into the disc space. In some embodiments, the method can further include delivering a third pedicle screw into the pedicle of the first vertebra on a contralateral side relative to the first pedicle screw, and delivering a fourth pedicle screw into the pedicle of the second vertebra on a contralateral side relative to the second pedicle screw. In some embodiments, the disc space further can be distracted at least in part by moving screw extensions engaged with at least two pedicle screws. In some embodiments, the screw extensions can be minimally invasive access towers. In some embodiments they can be retractor blades. In some embodiments the screw extensions can be integral to or attach to the housing of the pedicle screw. In some embodiments, the disc space can be distracted by simultaneously moving the retractor blades and screw extensions.

In another embodiment, a minimally invasive surgical method for treating the spine is provided. The method comprises delivering a first bone anchor into a first vertebra and a second bone anchor into a second vertebra, wherein the first bone anchor comprises a first shank and a first housing having a first extended tab and a first height, and wherein the second bone anchor comprises a second shank and a second housing having a second extended tab and a second height. A first portion of the first extended tab is removed such that the first housing has a third height less than the first height. A first portion of the second extended tab is removed such that the second housing has a fourth height less than the second height. A second portion of the first extended tab is removed after removing the first portion of the first extended tab, such that the first housing has a fifth height less than the third height. A second portion of the second extended tab is removed after removing the first portion of the second extended tab, such that the second housing has a sixth height less than the fourth height.

In some embodiments of the above method, removing a first portion of the first extended tab may comprise breaking the first portion off of the first extended tab. The third height may be equal to the fourth height. The fifth height may be equal to the sixth height. The method may further comprise positioning an attachment assembly over the first extended tab before removing the first portion of the first extended tab. The attachment assembly is a blade assembly. Removing the second portion of the first extended tab may comprise breaking the second portion off of the first extended tab. The method may further comprise inserting a surgical device into a working space before removing the second portion of the first extended tab and the second portion of the second extended tab, wherein at least a portion of the working space is between the first extended tab and the second extended tab. The surgical device may be an implant. The surgical device may be a tool. The first housing and/or the second housing may be monolithic.

In another embodiment, a minimally invasive surgical system for treating the spine is provided. The system comprises at least one bone anchor comprising a shank and a housing having an extended tab, the housing having a first height from a distal tip of the housing to a proximal tip of the housing, the extended tab comprising a plurality of frangible sections. The system also comprises an attachment assembly comprising an internal longitudinal bore sized to fit over at least a portion of the extended tab of the at least one bone anchor. The system also comprises a retractor assembly comprising a retractor arm configured to attach to the attachment assembly.

In some embodiments of the above system, the attachment assembly may have a second height from a distal tip of the attachment assembly to a proximal facing surface of the attachment assembly, the second height being less than the first height. The retractor arm may attach directly to the attachment assembly. The extended tab may comprise two prongs defining a transverse channel between them. The attachment assembly may comprise an inward projection configured to fit within at least a portion of the transverse channel. The system may further comprise a sleeve configured to be placed around a portion of the extended tab of at least one of the at least one bone anchors. The sleeve may be configured for placement between the extended tab of the at least one of the at least one bone anchors and an attachment assembly. The attachment assembly may comprise a lock that has a locked position and an unlocked position, and the lock in the locked position prevents longitudinal movement of the attachment assembly relative to the extended tab.

In another embodiment, an extended tab pedicle screw is provided. The pedicle screw comprises a screw shank and a housing attached to the screw shank. The housing comprises a plurality of frangible sections at different heights along the housing and two opposed side walls that define a transverse channel between them, the housing having a distal end nearest the screw shank and an opposite proximal end.

In some embodiments of the above pedicle screw, the screw shank may be cannulated. The plurality of frangible sections may be separated by scores on the side walls. The housing may further comprise a plurality of locking notches positioned longitudinally along the housing. At least two of the locking notches may be different sizes. A first locking notch may be distal to and wider than a second locking notch. Each locking notch may be wider than a more proximal locking notch or narrower than a more distal locking notch. The locking notches may also be identical.

In another embodiment, a minimally invasive surgical method for treating the spine of a patient is provided. The method comprises inserting a first screw into a first vertebra and a second screw into a second vertebra on an ipsilateral side of the patient, the first and second screws each comprising a shaft, a base portion and an extended tab portion comprising two tabs and a channel between the tabs, the extended tab portions extending above a skin level of the patient after the first and second screws are inserted. The method further comprises inserting a third screw into the first vertebra and a fourth screw into the second vertebra on a contralateral side of the patient, the third and fourth screws each comprising a shaft, a base portion and an extended tab portion comprising two tabs and a channel between the tabs, the extended tab portions extending above a skin level of the patient after the third and fourth screws are inserted. The method further comprises positioning blade assemblies over the first and second screws on the ipsilateral side, wherein the extended tab portions of the first and second screws extend above the upper surfaces of the blade assemblies. The method further comprises positioning one or more sleeves over the third and fourth screws on the contralateral side. The method further comprises attaching a retractor assembly to the blade assemblies and the one or more sleeves. The method further comprises removing upper portions of the extended tab portions of the first and second screws with remaining tab portions of the first and second screws positioned within the blade assemblies. The method further comprises distracting a disc space between the first and second vertebrae using the retractor assembly. The method further comprises inserting an interbody cage into the disc space on the ipsilateral side of the patient. The method further comprises removing the retractor assembly from the blade assemblies and sleeves. The method further comprises, after removing the blade assemblies from the first and second screws, inserting a first rod into the base portions of the first and second screws. The method further comprises, after removing the sleeves from the third and fourth screws, inserting a second rod into the base portions of the third and fourth screws. The method further comprises removing the remaining tab portions of the first and second screws from the base portions of the first and second screws. The method further comprises removing the extended tab portions of the third and fourth screws from the base portions of the third and fourth screws.

In some embodiments of the above method, the first and second screws may each comprise a plurality of frangible or weakened sections, and wherein removing the upper portions of the extended tab portions of the first and second screws and removing the remaining tab portions of the first and second screws from the base portions of the first and second screws causes separation along the frangible or weakened sections. The third and fourth screws may each comprise a frangible or weakened section between the extended tab portion and the base portion, and removing the extended tab portions of the third and fourth screws from the base portions of the third and fourth screws causes separation along the frangible or weakened section. The retractor assembly may attach to the blade assemblies through openings in arms of the retractor assembly. The retractor assembly may attach to the one or more sleeves with tubes attached to arms of the retractor assembly. Removing the upper portions of the extended tab portions of the first and second screws may occur before distracting the disc space. The method may further comprise delivering set screws through the channels between the tabs of the screws, the set screws engaging threads in the base portions of the screws to fix the rods in place. The method may further comprise positioning a medial blade between the blade assemblies of the first screw and the second screw.

In another embodiment, a minimally invasive surgical system for treating the spine is provided. The system comprises first and second bone anchors each comprising a threaded shaft, a base portion configured to receive a rod, and an extended tab portion comprising two tabs and a channel between the tabs, wherein the first and second bone anchors each further comprises a frangible or weakened section between the base portion and the extended tab portion, and the extended tab portion comprising a plurality of additional frangible of weakened sections. The system further comprises third and fourth bone anchors each comprising a threaded shaft, a base portion configured to receive a rod, and an extended tab portion comprising two tabs and a channel between the tabs, wherein the third and fourth bone anchors each further comprises a frangible or weakened section between the base portion and the extended tab portion. The system further comprises first and second blade assemblies each having a central bore configured to slide over the extended tab portions of the first and second bone anchors, respectively, wherein the first and second blade assemblies have a length that is shorter than a length of the extended tab portions. The system further comprises first and second sleeves each having a central bore configured to slide over the extended tab portions of the third and fourth bone anchors, respectively. The system further comprises a retractor assembly comprising a first arm and a second arm extending from a bar, wherein at least one of the arms is configured to move relative to the bar to change a distance between the arms. The first blade assembly and the first sleeve are configured to be attached to the first arm and the second blade assembly and the second sleeve are configured to be attached to the second arm.

In some embodiments of the above system, the arms may each comprise a first section and a second section that can articulate relative to each other. Each of the arms may comprise a collar configured to fit over a corresponding post of the first and second blade assemblies. The system may further comprise first and second tube assemblies attached to the first and second arms, respectively, wherein each tube assembly comprises a tube configured to slide over one of the first and second sleeves. The first and second blade assemblies may each further comprise an interior projection configured to fit between opposing tabs of the extended tab portions of the first and second bone anchors. The first and second blade assemblies may each further comprise wing or blade extensions extending from a central section of each blade assembly. The first and second blade assemblies may each further comprise a locking surface configured to engage notches on the first and second bone anchors. The system may further comprise a medial blade assembly attached to one or both arms, the medial blade assembly comprising a medial blade configured to be positioned between the first and second blade assemblies.

In another embodiment, a minimally invasive surgical system for treating the spine is provided. The system comprises a retractor assembly comprising a first arm and a second arm extending from a bar, wherein at least one of the arms is configured to move relative to the bar to change a distance between the arms. The system further comprises first and second blade assemblies configured to engage a first bone anchor inserted into a first vertebra and a second bone anchor inserted into a second vertebra, respectively, on an ipsilateral side of a patient, wherein the first blade assembly is configured to be attached to the first arm and the second blade assembly is configured to be attached to the second arm. The system further comprises an attachment assembly attached to the first and second arms and configured to connect to extensions extending from third and fourth bone anchors inserted into the first and second vertebrae, respectively, on a contralateral side of the patient. The blade assemblies are configured to engage the first and second bone anchors by sliding over extended tab portions of the first and second bone anchors.

In the embodiment of the system above, the system may further comprise first and second bone anchors each comprising a threaded shaft, a base portion configured to receive a rod, and an extended tab portion comprising two tabs and a channel between the tabs. The first and second bone anchors may each further comprise a frangible or weakened section between the base portion and the extended tab portion, and the extended tab portion comprising a plurality of additional frangible of weakened sections. The first and second blade assemblies may each further comprise an interior projection configured to fit between opposing tabs of the extended tab portions of the first and second bone anchors. The attachment assembly may comprise tubes configured to slide over the extensions extending from the third and fourth bone anchors. The system may further comprise third and fourth bone anchors each comprising a threaded shaft, a base portion configured to receive a rod, and an extended tab portion comprising two tabs and a channel between the tabs. The third and fourth bone anchors may each further comprise a frangible or weakened section between the base portion and the extended tab portion. The arms may each comprise a first section and a second section that can articulate relative to each other. Each of the arms may comprise a collar configured to fit over a corresponding post of the first and second blade assemblies. The first and second blade assemblies may each further comprise wing or blade extensions extending from a central section of each blade assembly. The first and second blade assemblies may each further comprise a locking surface configured to engage notches on the first and second bone anchors. The system may further comprise a medial blade assembly attached to one or both arms, the medial blade assembly comprising a medial blade configured to be positioned between the first and second blade assemblies.

### BRIEF DESCRIPTION OF THE DRAWINGS

FIG. 1 is a perspective view of one embodiment of a retractor system for use in a minimally invasive surgical system.

FIG. 2 is a perspective view of one embodiment of a pedicle screw with a rod and set screw.

FIG. 3 is a perspective view of one embodiment of a retractor blade of a retractor blade assembly.

FIG. 4 is a cross-sectional side view of the retractor blade of FIG. 3.

FIG. 5 is a perspective view of one embodiment of a shaft of a retractor blade assembly.

FIG. 6 is a cross-sectional side view of the shaft of FIG. 5.

FIG. 7 is a perspective view of one embodiment of a retractor blade assembly.

FIG. 8A is a top cross-sectional view of the retractor blade assembly of FIG. 7.

FIG. 8B is a perspective view of the retractor blade assembly of FIG. 8A, with the retractor blade transparent.

FIG. 9A is a side view of one embodiment of a screw extender.

FIG. 9B is a side view of the screw extender of FIG. 9A, rotated 90 degrees.

FIG. 9C is a cross-sectional view of the screw extender of FIG. 9A.

FIG. 10 is a side view of one embodiment of a screwdriver.

FIG. 11 is a top cross-sectional view of one embodiment of a screw extender.

FIG. 12 is a side view of one embodiment of a screwdriver.

FIG. 13A is a top view of one embodiment of a locking plate.

FIG. 13B is a side cross-sectional view of the locking plate of FIG. 13A.

FIG. 14 is a cross sectional view of a section of a screwdriver and screw extender that can lock a screwdriver on two sides.

FIG. 15 is a perspective view of one embodiment of a retractor system showing one embodiment of an extension attachment assembly of FIG. 1.

FIG. 16 is a perspective view of one embodiment of a retractor system showing one embodiment of a medial blade attachment assembly of FIG. 1.

FIG. 17 is a top view of one embodiment of a retractor system showing one embodiment of the extension attachment assembly and the extension attachment assembly of FIG. 1.

FIGS. 18-20 are perspective views of one embodiment of a medial blade.

FIGS. 21-22 are perspective views of another embodiment of a medial blade and a light wand tool assembly.

FIGS. 23A-B are perspective views of a spacing tool.

FIG. 24 is a perspective view of one embodiment of a minimally invasive tower access device.

FIGS. 25-27 perspective views of one embodiment of a retractor system for use in a minimally invasive surgical system.

FIG. 28 is a perspective view of one embodiment of a retractor system for use in a minimally invasive surgical system.

FIGS. 29A-B are front and side views, respectively, of one embodiment of a bone screw.

FIG. 29C is a cross-section of the bone screw of FIG. 29B.

FIG. 30A is a front view of one embodiment of a blade assembly for use in a minimally invasive surgical system.

FIG. 30B is a cross-section of the blade assembly of FIG. 30A.

FIGS. 31A-B are top and bottom views, respectively of the blade assembly of FIG. 30A.

FIGS. 32A-B are top and side views, respectively of one embodiment of a locking button.

FIG. 33 is a top view of one embodiment of a sleeve for use with a minimally invasive surgical system.

FIG. 34A is a front view of the sleeve of FIG. 33.

FIG. 34B is a cross section of the sleeve of FIG. 34A.

FIG. 35 is a top view of one embodiment of a sleeve for use with a minimally invasive surgical system.

FIG. 36 is a cross-section of the sleeve of FIG. 35.

FIG. 37 is a plurality of bone screws inserted into pedicles of a patient's spine.

FIG. 38 shows instruments inserted over the bone screws of FIG. 37.

FIG. 39 shows a retractor body positioned relative to the instruments of FIG. 38.

FIG. 40 shows one embodiment of an attachment assembly used to attach a screw to the retractor body of FIG. 39.

FIG. 41 shows a transverse plane view of the assembly of FIG. 40.

FIG. 42 shows the assembly of FIG. 40 with a portion of two bone screws removed.

FIG. 43 shows a plurality of bone screws positioned into pedicles of a patient's spine with a portion of two bone screws removed.

### DETAILED DESCRIPTION OF THE PREFERRED EMBODIMENTS

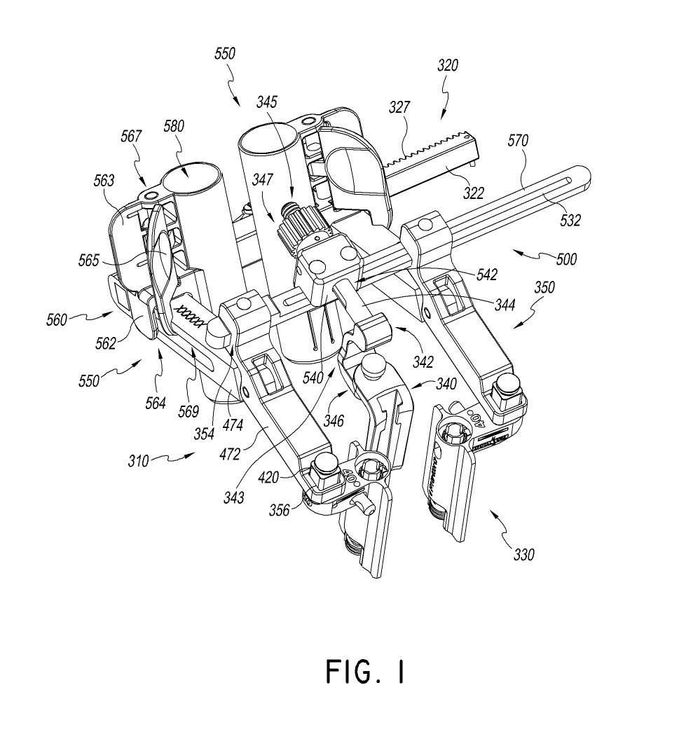

FIG. 1 illustrates an embodiment of a retractor system 310 used as part of a minimally invasive (MIS) and/or maximum access (MAS) spinal surgery. This disclosure may refer to MAS or MIS surgeries with respect to certain embodiments, but it is to be understood that such use does not preclude any embodiment described herein from being considered an MIS and/or an MAS surgery.

In some embodiments, the minimally invasive (MIS) and maximal access (MAS) surgical techniques and apparatuses disclosed herein can include a retractor body 320 with a plurality of arms 350. The device as illustrated has two arms extending from a cross bar 322 of the retractor body. Generally, during a surgical procedure the cross bar 322 can be positioned above a patient's back and the arms 350 can point laterally on an ipsilateral or operational side of the patient's spine. As used throughout the present disclosure, the “operational side” shall refer to a side of the patient from which disc space of the spine is accessed (e.g., for delivery of a spinal implant). In some embodiments, the cross bar 322 can be positioned generally directly above the patient's spine or on the operational side of the patient's spine and the arms can point laterally. In some embodiments, the cross bar 322 can be positioned on a contralateral side (i.e., a side opposite the operational side) of the patient's spine and the arms can extend across the spine. In some embodiments, the cross bar 322 can be positioned above or adjacent the spine and the retractor body can have two arms extending laterally on the contralateral side and two arms extending laterally on the operational side, for a total of four arms. In some embodiments, the retractor body can have more than two arms on one or both sides.

The arms 350 can be configured to attach to anchor extensions, such as lateral blade assemblies 330, which can each be configured to attach to and extend from a bone anchor (typically a bone screw, such as a pedicle screw). Bone screws can attach to blade assemblies such that the shaft of the screw extends below the bottom edge of the blade. “Bottom” or “below” as used herein are with reference to the anterior side of the patient when the disclosed devices and components are positioned on or in the patient during posterior surgery. Thus, the bottom edge of a blade assembly 330 is the edge that would face the anterior side of the patient. The lateral blade assemblies 330 can be used to retract tissue during a surgical procedure. Additionally, because the lateral blades are attached to the pedicle screws, and because they can be configured to move with the arms in either direction along a generally caudal-cranial line when positioned in a patient (as described further below), they can also help distract disc space when the pedicle screws are screwed into adjacent vertebrae. Other types of anchor extensions can similarly be used to distract a disc space.

In some embodiments, a medial blade assembly 500 can be attached to the retractor body 320, such as to one or more of the arms 350. The medial blade 340 can be configured to move in a medial-lateral direction when placed within a patient, helping to retract tissue and create a clear view of the vertebrae. In some embodiments, the medial blade assembly can attach to the arms through attachment openings 354. As described in more detail below, the medial blade assembly can include an assembly attachment portion 510, a medial blade positioning portion 540, and a medial blade arm 342 that can attach to a medial blade 340.

In some embodiments, a retractor system can also include one or more anchor extension attachment assemblies 550. The extension attachment assemblies can be used to attach the retractor system to a variety of anchor extensions or surgical devices, such as one or more minimally invasive surgical (MIS) towers, as discussed further below. In some embodiments, a retractor body 320 can be positioned such that lateral blade assemblies 330 are on one side (e.g., an operational side) of a patient's spine and extension attachment assemblies 550 are on another side (e.g., a contralateral side) of the patient's spine.

Where the lateral blade assemblies are attached to pedicle screws on the operational side and the extension attachment assemblies interface with extensions (such as MIS towers) attached to pedicle screws on the contralateral side, separating the arms 350 of the retractor body 320 can create bilateral distraction, distracting the disc space on both sides of the spine. In some embodiments, the arms can be generally parallel to each other, such that the bilateral distraction creates a generally equal amount of distraction on both sides of the spine. In some embodiments, the arms can be at an angle relative to one another, such that bilateral distraction creates an amount of distraction on a first side of the spine that differs from the amount of distraction on a second side of the spine.

Having the retractor system 310 attached to anchor extensions on both sides of the spine creates a number of advantages. For example, the retractor system will be better anchored into the body. In some embodiments, the anchoring can be such that the system does not need to be anchored to the operating table or other external support structure, as is typically done. Further, where two screws are inserted into the operational side (one each on two adjacent vertebrae) and corresponding screws are inserted on the contralateral side, the retractor system can distribute forces between four screws instead of just two. Additionally, by allowing for distraction on both sides of the disc space, the retraction system described herein can be used to perform a TLIF, TPLIF, PLIF or other procedure according to the surgeon's choice without having to vary the setup.

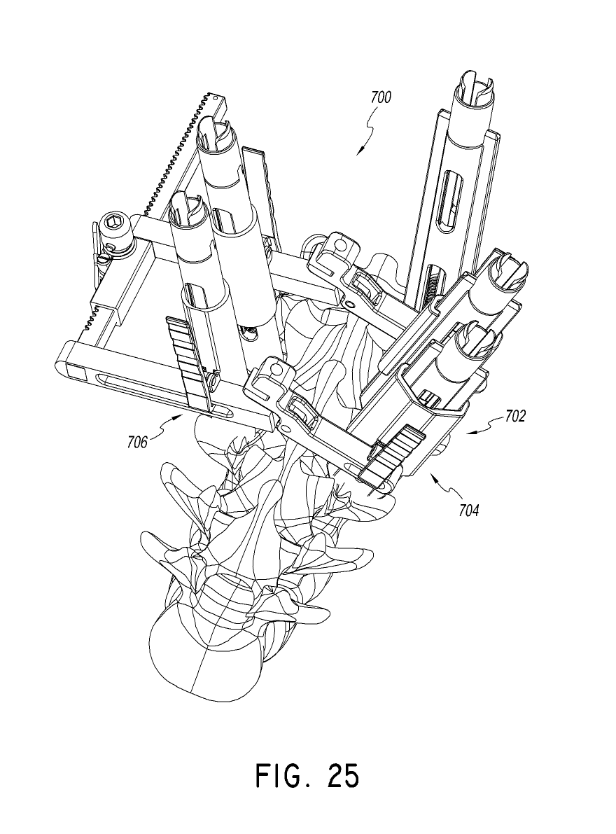

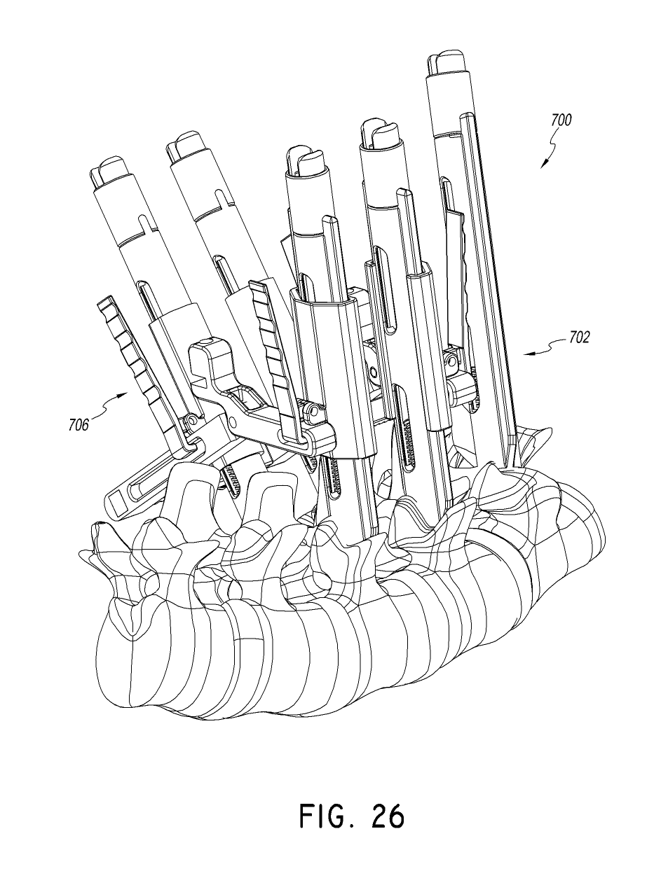

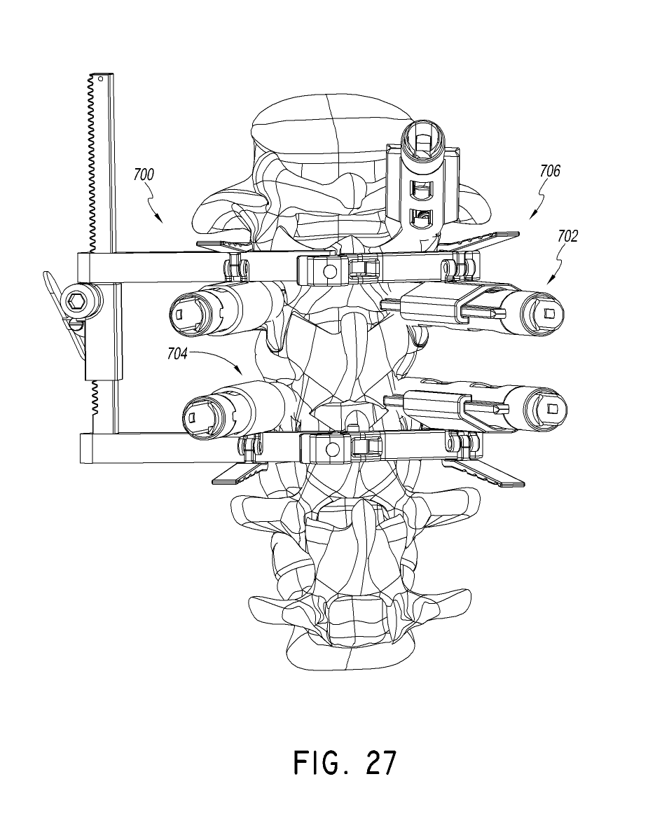

In some embodiments, a surgeon can modify the device from the illustrated embodiment according to preference. For example, in some embodiments a surgeon may attach four extension attachment assemblies 550 to the arms 350 of the device. This could allow, for example, the surgeon to attach the retractor body to MIS towers on an operational side of a patient's spine and to MIS towers on a contralateral side of the patient's spine. Alternatively, in some embodiments a surgeon may elect to have two lateral blade assemblies on a first side of the spine and a lateral blade assembly and an extension attachment assembly on a second side of the spine. In some embodiments, the device may have four lateral blade assemblies. Alternate configurations may include more than two blade assemblies or extension attachment assemblies on one or both sides of the spine. According to another embodiment, shown for example in FIGS. 25-27, a retractor system for use in a minimally invasive surgical system is configured to be used with extended tab percutaneous screws.

With reference to FIG. 1, the extension attachment assembly 550 can include a tube assembly 580. In some embodiments, the tube assembly can be configured to connect to an anchor extension at a fixed angle relative to the arms 350. In some embodiments, the tube assembly can be configured to connect to an anchor extension at varying angles relative to the arms 350. Additionally, as discussed further below, in some embodiments the extension attachment assemblies 550 can be configured to move along a length of the arm 350 into a desired position.

In some embodiments, it can be desirable to attach devices that allow for improved visibility of a surgical location. In some embodiments, lighting components 302 can attach to a retractor system 310 at any location that helps provide light to the surgical location without unduly obstructing the surgeon's view. For example, in some embodiments lighting components 302 can attach to the medial blade 340. Lighting components can attach to other portions of the retractor system 310 as well.

In some embodiments the retractor arms 350 can have a lateral blade connection assembly or collar 356, which can be used to attach the arms to a lateral blade assembly. The collar can have a variety of shapes. In the illustrated embodiment, the collar is configured to attach to a rectangular post of a lateral blade assembly, discussed further below. This can help ensure that the blade assembly is properly oriented when attached to a retractor arm 350. FIG. 1 also illustrates an embodiment of a retractor system in which the extension attachment assemblies 550 are not aligned. Thus, a first attachment assembly can be positioned a first distance from the crossbar 322 and a second attachment assembly can be positioned a second distance from the crossbar 322, such that the first distance is different from the second distance. In some embodiments, the two attachment assemblies can be positioned the same distance from the cross bar 322. In such embodiments, a line perpendicular to both arms 350 can pass through a center of both tube assemblies 580. In some embodiments, one or more retractor arms 350 can have attachment holes, while in some other embodiments, one or more retractor arms 350 can have no attachment holes.

### Pedicle Screws

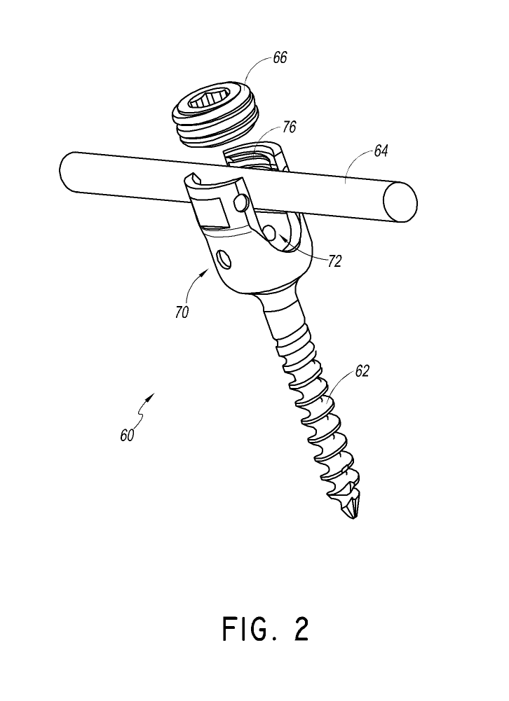

FIG. 2 illustrates a pedicle screw 60 that can be used with a MAS retractor system, as disclosed herein. The pedicle screw can include a cannulated shaft 62 (such that the screw can follow a guide wire) and a housing 70 (also referred to as a head or tulip). The housing can have interior threading 76 and openings 72 extending from the top of the housing on opposite sides. The openings can be sized to receive a rod 64, which can be used to connect multiple pedicle screws and create a spine stabilization system, and which can be locked into place by a set screw 66. Further details of pedicle screws are provided in U.S. Patent Application Publication No. 2010/0241175, published on Sep. 23, 2010, which is hereby incorporated by reference in its entirety and is to be considered part of this specification.

### Retractor Blade Assemblies

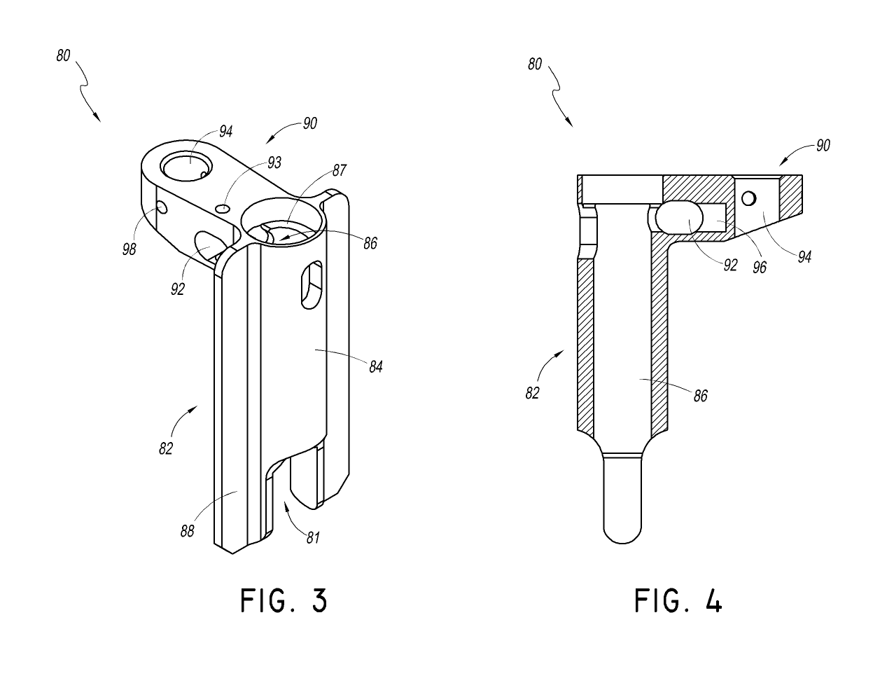

FIGS. 3 and 4 illustrate one embodiment of a retractor blade 80 that can be used with a MAS retractor system as disclosed herein. FIG. 3 is a perspective view of the blade, and FIG. 4 is a cross-sectional view. The retractor blade can comprise an attachment section 90 and a blade section 82, the blade section extending down from the attachment section. The blade section can have a cylindrical central bore 86 within a generally cylindrical central section 84, and the central bore can run from the top of the retractor blade through the bottom of the central section. In some embodiments, the top of the bore can have a larger diameter than lower sections of the bore, creating a ledge 87. In some embodiments, the ledge 87 can be a ramped surface. In some embodiments, the generally cylindrical central section 84 can be square, oval, or of other shapes.

The blade section 82 can have wings or blade extensions 88 that extend outward from opposite sides of the central section 84. The blade extensions 88 can extend below a bottom most point of the central section 84, creating a gap 81 as illustrated. In some embodiments, the blade extensions can extend straight out from the central section 84 in a common, shared plane. In some embodiments, the blade extensions can be curved and/or extend out at an angle from the central section. The blade extensions are preferably symmetric about a line perpendicular to the longitudinal axis of the central bore 86 of the blade 80. For reference purposes, the plane that is perpendicular to the blade extension line of symmetry, and on which the longitudinal axis of the central bore lies, can be referred to as the “blade plane.” In some embodiments, there are no blade extensions extending outward from the central section 84 and the central sections of adjacent retractor blades can provide access to a surgical site, with or without a medial blade assembly 42.

The attachment section 90 can extend from the blade section 82 and can be formed integrally with the blade section. Preferably, the attachment section extends from the blade section in a direction perpendicular to the blade plane. In some embodiments, the attachment section can have a lever bore 92 that extends at least partially into the attachment section from the same side as one of the blade extensions 88. The attachment section can also have a pin bore 96, which can extend into the attachment section from the central bore 86. In some embodiments, a longitudinal axis of the pin bore can be orthogonal to a longitudinal axis of the central bore. In some embodiments, a longitudinal axis of the lever bore 92 can be orthogonal to both the longitudinal axis of the pin bore 96 and the longitudinal axis of the central bore 86. The attachment section can also include a post attachment hole 94, a post pin hole 98, and a lever pin hole 93, as illustrated.

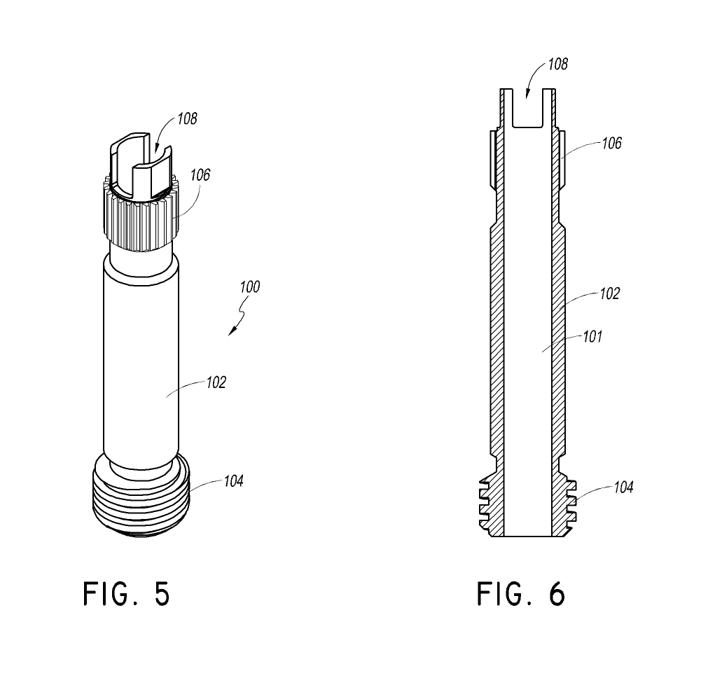

FIGS. 5 and 6 illustrate a retractor blade shaft 100, which can be inserted into the central bore 86 of the retractor blade 80. FIG. 5 is a perspective view of the shaft 100 and FIG. 6 is a cross sectional view. The blade shaft 100 can include a middle shaft section 102, a threaded portion 104 along a bottom section of the retractor blade shaft, and a central shaft bore 101 that extends through the entire length of the shaft. The threaded portion 104 is preferably sized to be able to screw into the tulip of a pedicle screw, mating with the interior threads of the tulip. The retractor blade shaft 100 can also include a plurality of vertical locking grooves 106 extending around the shaft at an upper end thereof. Above the locking grooves 106, the retractor blade shaft can also have a plurality of notches 108, which can be spaced on opposite sides from each other. In some embodiments, the section of the shaft with locking grooves 106 can have substantially the same diameter as the middle shaft section 102. In some embodiments, the middle shaft section 102 and the section with locking grooves 106 can each be sized and configured to fit within the central bore 86 of the retractor blade 80.

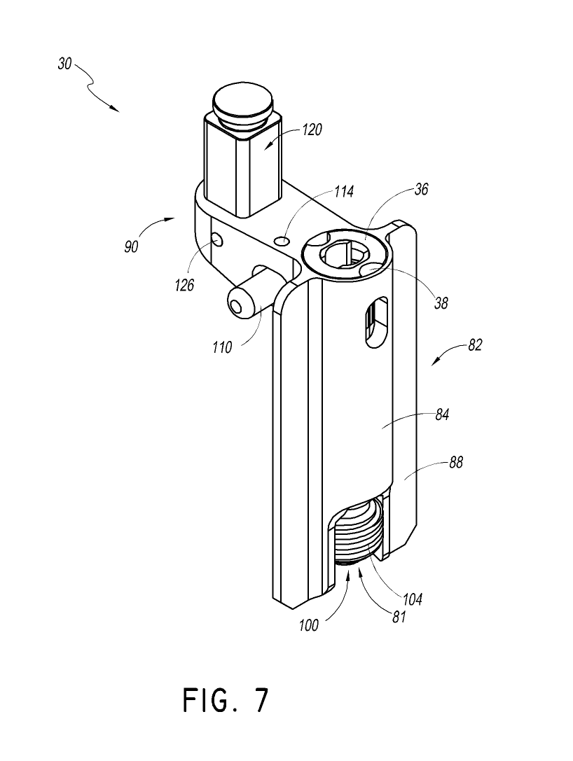

FIG. 7 illustrates a perspective view of a lateral blade assembly 30 in which the retractor blade shaft 100 has been positioned within the central bore of the retractor blade 80. Although not visible in FIG. 7, when the blade shaft is within the retractor blade at least a portion of the locking grooves 106 can be level with at least a portion of the attachment section 90. In some embodiments, the threaded portion 104 of the blade shaft can be wider than the central bore 86, such that to be assembled the retractor blade shaft must be inserted from the bottom of the central bore. The threaded portion 104, however, is preferably sized such that it can fit within the gap 81 of the retractor blade. In some embodiments, the retractor blade shaft 100 has a length such that the threaded portion 104 does not extend all the way to the bottom of the blade extensions 88 when the retractor blade shaft is positioned within the retractor blade 80.

Preferably, the retractor blade 80, the retractor blade shaft 100, and a pedicle screw 60 are all sized and configured such that the threaded bottom 104 of the blade shaft can be screwed into the tulip 70 of the pedicle screw. The blade extensions 88 can be sized such that as the blade assembly 30 is screwed into the tulip the extensions slide into the openings 72 along the sides of the tulip (visible in FIG. 2). This can prevent the tulip from rotating relative to the retractor blade 80.

Because the blade assembly 30 can attach directly to the tulip 70 of the pedicle screw 60, the screw does not have to be installed in a patient in a modular fashion. Consequently, in some embodiments the screw can be installed in a patient as a preassembled or non-modular unit, saving the extra steps of attaching the tulips after performing or during the desired procedure. Further, in some embodiments the retractor blade shaft 100 can rotate within the central bore 86, allowing the blade assembly 30 to be screwed into the tulip 70 of a pedicle screw 60 without rotating the retractor blade 80 itself. Thus, in some embodiments the blade assembly 30 and pedicle screw 60 can be screwed together after the pedicle screw is placed within a vertebra. Further, because the blades can rotate, a single blade can be used regardless of the side of the disc space to which the blade is attached; if the blade needs to be rotated for proper positioning it can easily do so. This also can make it easier to perform surgical procedures on multiple spinal levels, as discussed further below.

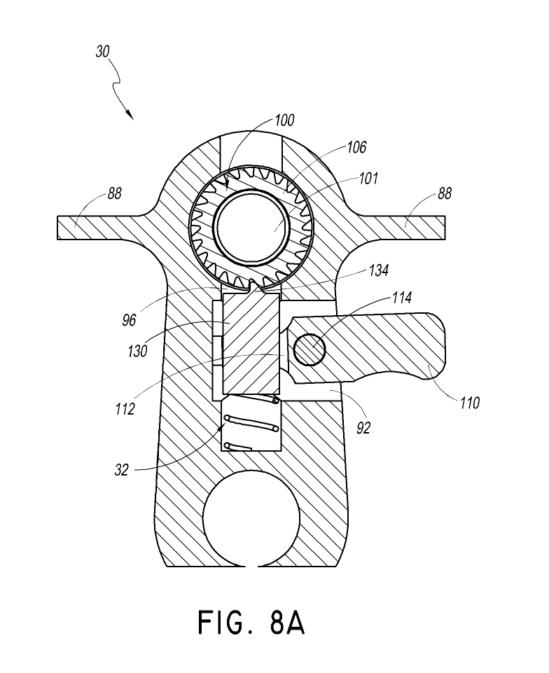

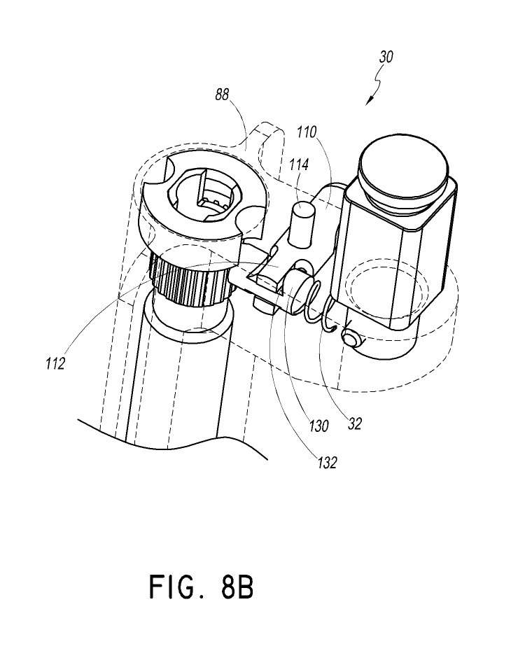

FIGS. 8A and 8B illustrate a mechanism that can prevent the retractor blade shaft 100 from unscrewing itself from the tulip of a pedicle screw. FIG. 8A is a top cross-sectional view of the lateral blade assembly 30, and FIG. 8B is a perspective view of the assembly with the retractor blade transparent for visibility. The blade assembly can include a locking pin 130 positioned at least partially within the pin bore 96. The locking pin can have a point 134 on one end that can fit within the locking grooves 106. A spring 32, also positioned within the pin bore 96, can bias the locking pin 130 into a locked position, in which the point 134 of the pin is in locking engagement with the locking grooves 106. In some embodiments, the locking grooves 106 can be angled relative to a diameter of the shaft bore 101 in order to create a ratcheting effect, such that when the pin is in a locked position the shaft 100 can rotate in a direction to screw into a tulip of a screw (typically clockwise), but is prevented from rotating in the opposite direction.

In some embodiments, the blade assembly 30 can have a lever 110 that can be used to manually release the locking pin 130 from the locked position, thus allowing the retractor blade shaft 100 to unscrew from its position within a pedicle screw. As illustrated in FIG. 8A, the lever 110 can be positioned within the lever bore 92. The lever bore can have an oblong shape, or at least shape that is larger than the lever 110 such that the lever can move within the lever bore. The locking pin 130 can have one or more gaps 132 (visible in FIG. 8B) bounded on either side by a surface, and an end 112 of the lever can be positioned within the gap. In the illustrated embodiment, the locking pin has a gap on an upper and a lower side of the locking pin, and the end of the lever is split into two prongs, one positioned within each gap. In some embodiments, the surfaces on either side of the gap can be curved.

The lever can be held in position by the lever pin 114, about which the lever can rotate. Consequently, pushing the lever toward the blade extensions 88 will cause the end of the lever 112 to rotate toward the spring 32. The end of the lever will push against a surface bounding a gap (or gaps) 32 of the locking pin 130 and push it toward the spring, releasing the locking pin 130 from the locked position and moving it into an unlocked position in which the retractor blade shaft 100 can freely rotate in either direction and can be unscrewed from the pedicle screw. When the lever is released the spring will tend to push the locking pin back into the locked position.

Returning to FIG. 7, a post 120 can be attached to the attachment section 90 of the retractor blade 80, typically on an upper surface of the attachment section. In some embodiments, the post can be inserted directly into the post attachment hole and held in place with a pin 126. The post can be used to attach the blade assembly 30 to the retractor, as discussed below. In some embodiments, the post can have a variety of shapes. In some embodiments, as illustrated, it can be generally rectangular. In some embodiments, the post can be generally cylindrical, oval, or of other shapes. In some embodiments, the post can have one or more detents to help align the post and blade assembly as desired.

Also visible in FIG. 7 is a cap 36, the top surface of which can be donut-shaped with a hole passing through its center, although the hole can be of any desired shape. The cap can be positioned in the central bore 86 around at least a portion of the retractor blade shaft 100, and can be attached (e.g., welded) onto the retractor blade shaft. In some embodiments, the hole in the cap can be aligned with the shaft bore 101. The outer perimeter of the cap can extend into the central bore 86 such that it touches or is adjacent to the ledge 87 (visible in FIG. 3), blocking the shaft from downward movement once the cap is attached to the shaft. So positioned, the top of the cap can be generally flush with an upper surface of the retractor blade 80. The cap can also have two curved cutouts 38 on opposite sides of each other. When the cap is attached to the retractor blade shaft, the cutouts 38 are preferably oriented at approximately 90 degrees from the notches 108 of the retractor blade shaft.

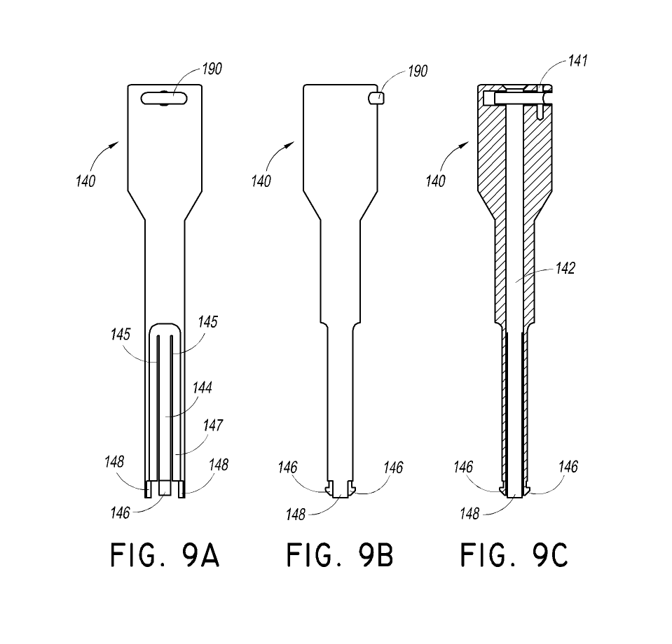

FIGS. 9A-11 illustrate a screw extender 140 and screwdriver 150. The screw extender can be used to attach the blade assembly to a pedicle screw, and the screwdriver can be used to insert the pedicle screw into a vertebra. As illustrated in FIG. 9A, a screw extender 140 can have at least two downward projections 148 that extend to the bottom of the screw extender opposite each other. The screw extender can also have two flexible sections 144, preferably positioned opposite each other as well, and preferably positioned such that each flexible section is located approximately 90 degrees about the screw extender from each downward projection. Each flexible section can be formed by a pair of cuts 145 on an outer surface 147 of the screw extender 140, the cuts extending along a portion of the length of the extender. In some embodiments, each pair of cuts can be parallel to each other.

FIG. 9B illustrates a side view of a screw extender 140, rotated 90 degrees from the view of FIG. 9A. FIG. 9C illustrates a cross-sectional view of FIG. 9B. As can be seen in FIG. 9B, the flexible sections 144 can have an equilibrium or locked position that is substantially flush with or angled slightly in from the outer surface 147 of the extender. However, the flexible sections can each have an outward extension 146 that extends beyond a plane of the respective outer surface 147 when the flexible sections are in the equilibrium or locked position. The flexible sections can be pushed inward into a central bore 142 (visible in FIG. 9C) until the flexible sections reach an unlocked position. In the unlocked position, the outward extensions 146 do not extend out past the planes of their respective outer surfaces 147.

When the flexible sections 144 are in the unlocked position, the screw extender can be inserted into the cap 36 of the blade assembly 30 (visible in FIG. 7). The downward projections 148 can fit within the curved cutouts 38, and the outward extensions 146 can fit within the central hole in the cap. Because the cap is oriented such that the cutouts are 90 degrees from the notches 108 of the shaft, and because the projections 148 are oriented 90 degrees from the outward extensions 146, the outward extensions will line up with the notches 108 of the shaft. When the flexible sections 144 are allowed to return to the equilibrium or locked position, the outward extensions will each extend into a notch 108 and below the top surface of the cap 36, thereby preventing removal of the screw extender 140 from its position in the cap. When the screw extender is in the cap, rotating the screw extender will rotate the retractor blade shaft 100, allowing it to be screwed into or out of the tulip of a pedicle screw.

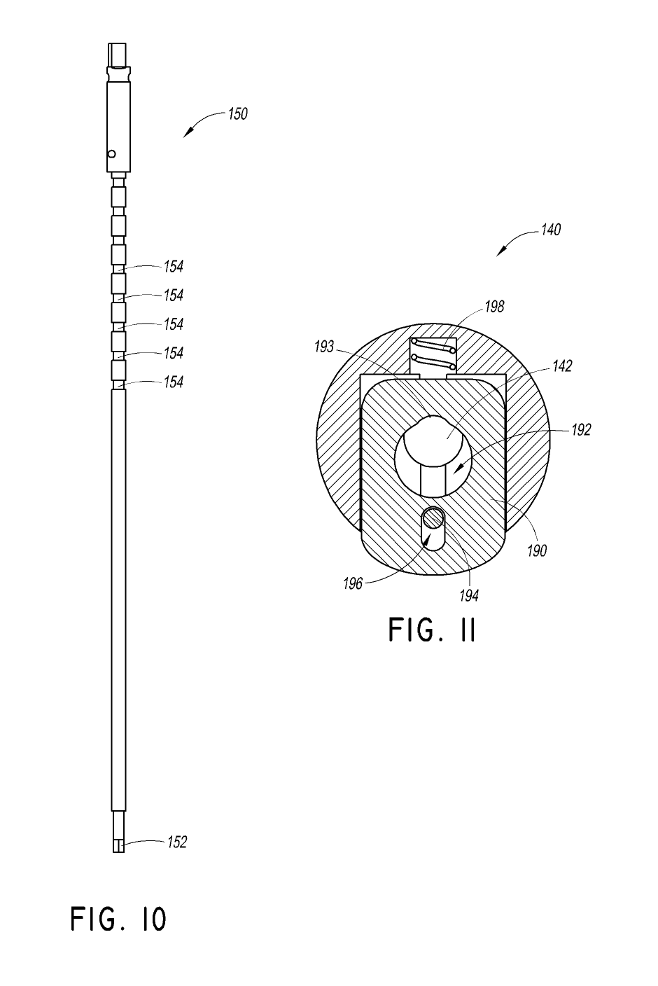

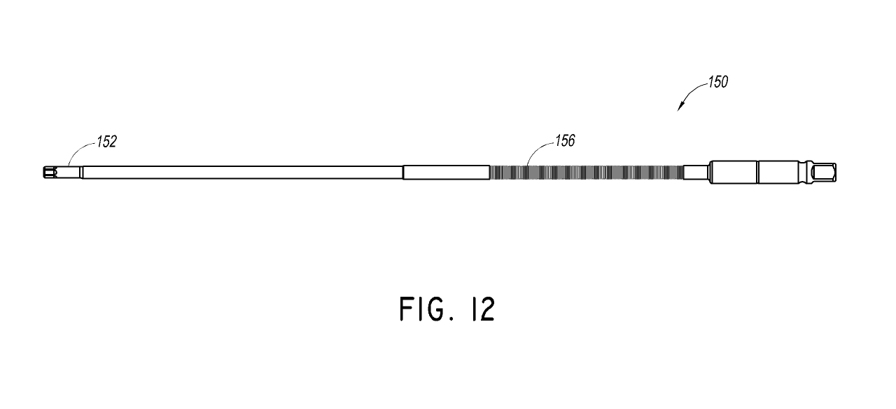

FIG. 10 is a side view of a screwdriver that can be used with the system described herein. The screwdriver is generally cylindrical, and is preferably sized such that it fits substantially flush within the central bore 142 of the screw extender 140. Thus, the screwdriver can occupy the available space within the central bore, preventing the flexible sections 144 from being pushed into the unlocked position. This can create a more solid connection between the screw extender and the blade shaft 100 while the screwdriver is being used.

The screw driver is preferably long enough to pass through the screw extender 140 and the bore 101 of the retractor blade shaft 100 in order to reach the shaft of a screw. The screwdriver has a distal tip 152 that can be configured to fit within a corresponding recess on the top of the screw shaft (e.g., a hex configuration), allowing the screwdriver to tighten the screw into a vertebra.

In some embodiments, it can be desirable to lock the screwdriver into a desired position within the screw extender 140 and blade shaft 100. This can be achieved by forming a plurality of annular recesses 154 on the outer surface of the screwdriver. The sections of the screwdriver with annular recesses can have a recessed diameter. The sections of the screwdriver between the annular recesses can have a standard diameter. The recesses can be used to lock the screwdriver against axial motion.

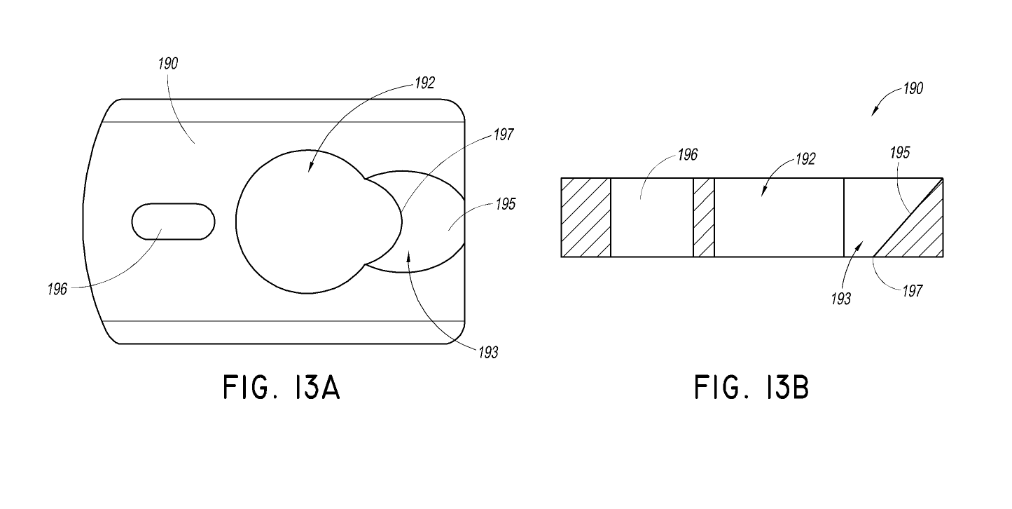

FIG. 11 is a top cross-sectional view of the screw extender 140 and illustrates a mechanism that can be used with the annular recesses to lock the screwdriver against axial motion. The screw extender can comprise a movable locking plate 190 with a generally circular cutout 192 and a positioning slot 196. The positioning slot can fit around a projection 194, which can help prevent the locking plate 190 from moving out of place. In some embodiments, the projection can be a pin inserted into a pin hole 141 (visible in FIG. 9C). In some embodiments, the cutout can have a section 193 with a smaller radius of curvature than the rest of the cutout.

The locking plate can block at least some of the central bore 142 of the screw extender. In an unlocked position, the locking plate is positioned such that at least sections of the screwdriver with the standard diameter can pass freely through. In a locked position, as illustrated in FIG. 11, the locking plate has blocked enough of the central bore 142 such that sections of the screwdriver with the standard diameter can no longer pass through.

In some embodiments, the screw extender can include a spring 198 or other biasing member, which can bias the locking plate into the locked position. Consequently, as the screwdriver is fed through the screw extender, when an annular recess passes through the cutout 192 of the locking plate 190 the locking plate will be pushed into the locked position. This will prevent further withdrawal or entry of the screwdriver, because the sections of standard diameter above and below the annular recess will be blocked by the locking plate.

The screwdriver can still rotate, however. In some embodiments, to improve the ability of the screwdriver to rotate when locked into the screw extender, the radius of curvature of the section 193 of the cutout can be approximately equal to half of the recessed diameter.

As visible in FIG. 11, and also FIG. 9B, the locking plate extends out of the interior of the screw extender, and can be manually pushed into the unlocked position. It can also be held in the unlocked position for easy insertion and removal of the screwdriver, or to position the screwdriver into a desired position before allowing the locking plate to lock the screw driver with a desired recess. The plurality of recesses can allow for locking the screwdriver in a desired position for use with blades and/or screw extenders of varying length.

In some embodiments, it can be desirable to have a screwdriver that has the ability for more refined axial locking. For example, in some embodiments, manufacturing tolerances in various components, such as the screw extender length, the depth of the recess in the screw, and/or the blade assembly length, can combine to make it such that the screwdriver does not properly engage the screw in any of its locked positions. This risk can be minimized by increasing the number of annular recesses in the screwdriver and decreasing their width.

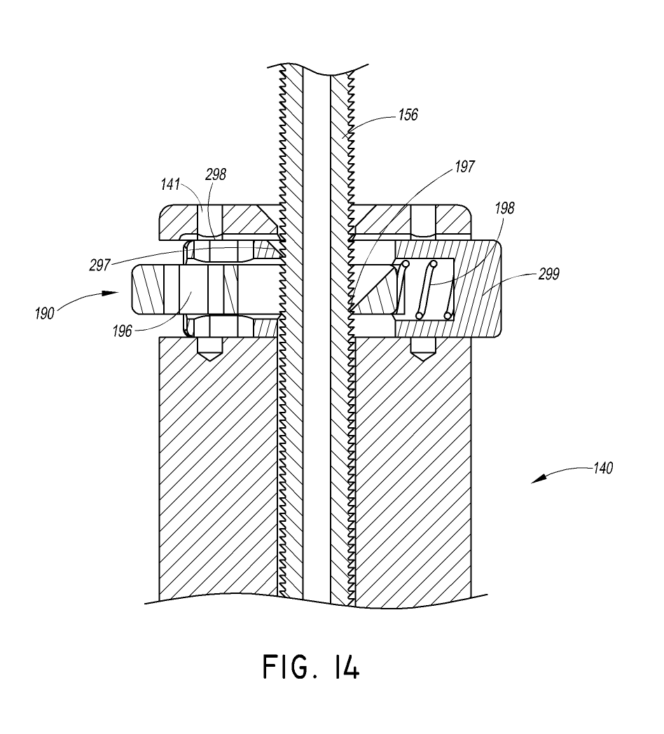

FIG. 12 illustrates one embodiment of a screwdriver that can minimize problems created from varying manufacturing tolerances. As illustrated, the screwdriver can have a section 156 with external ridges. The ridged section can help allow for a continuous span of lockable positions along the length of the ridged section. Thus, the screwdriver can be moved to adjust for any variance in height due to manufacturing tolerances and to ensure that the screwdriver is able to properly engage a screw. The screwdriver can also be adjusted for any variance in height requirements among different procedures. In some embodiments, the screwdriver can have a threaded section instead of or in addition to the section with external ridges.

The screwdriver of FIG. 12 can be used with screw extenders as described above, including use of a locking plate to engage the screwdriver as described with respect to FIG. 11. In some embodiments, the locking plate can be modified to match the screwdriver. For example, FIGS. 13A and 13B illustrate one embodiment of a locking plate 190 configured for use with a ridged screwdriver. FIG. 13A is a top view and FIG. 13B is a side cross-sectional view. The locking plate can have a scalloped or trimmed portion to create an edge or tooth 197 in the section 193 with a smaller radius of curvature than the rest of the cutout 192. The tooth 197 can be sized and angled to engage the ridged section 156 and to prevent axial translation of the screwdriver when engaged. Thus, the screwdriver may not slide axially when locked, but it can still rotate, which can allow it to drive a screw forward or backward.

In some embodiments, the locking plate 190 and/or screwdriver 150 can receive a coating to increase its hardness and wear resistance. Coatings can include, for example, CrN, TiN, ZrN, a-C:H, etc. This can increase the life of the screwdriver and of the locking plate.

In some embodiments, a screw extender can have more than one locking plate 190 to engage the threaded section 156 of the screwdriver. This can help provide a more stable connection between the screw extender and screwdriver, while also increasing the durability of the system by sharing loads among multiple components. In some embodiments, having one or more locking plates engage the screwdriver on opposite sides can also help minimize loading on components by balancing loads on the screwdriver. This can further help the driver remain concentric with respect to the components of the screw extender.

FIG. 14 illustrates a cross sectional view of one embodiment of a screw extender 140 that can lock the screwdriver by engaging it on multiple sides. The screw extender can include a locking plate 190 similar to the embodiment described with respect to FIGS. 13A and 13B. The locking plate can be positioned adjacent a spring 198 within a locking plate cover 299, which can also be configured to receive a screwdriver. Both the locking plate and locking plate cover can have slots 196, 298 respectively, which can receive a pin through a pin hole 141. This can allow the plate and cover to move within a range of positions relative to each other. In some embodiments, the locking plate cover can have an edge or tooth 297, similar to the edge or tooth 197 of the locking plate 190, and that generally faces the edge or tooth of the locking plate. The spring can be biased to move the locking plate and locking plate cover toward each other, which can push the teeth 197, 297 into engagement with the threaded section 156 of the screwdriver. A user can then push the locking plate and locking plate cover radially inward into the screw extender 140 to release the screwdriver and allow for its free axial motion.

Different arrangements are considered for engaging a screwdriver on multiple sides. For example, in some embodiments two separate springs can be used, each spring configured to engage a locking plate or a locking plate cover. In some embodiments, two locking plates can be positioned in the same plane on opposite sides of a screwdriver, and each locking plate can be spring biased into the screwdriver. Any other arrangement that biases a tooth into the screwdriver in more than one location can be used.

The various embodiments of screwdrivers and/or extenders described herein are not limited for use with a retractor system. They can be used in any surgical system that requires the use of a screwdriver and in which it may be beneficial to lock the screwdriver in a desired position. Such locking can, for instance, reduce the amount of toggle between the screw and screwdriver by ensuring a minimum engagement between the two. The various embodiments of screwdrivers and/or extenders described herein would have utility in any surgical system used to drive screws into bone, including but not limited to: pedicle screws such as for PLIF, TLIF, TPLIF and other surgeries; anterior cervical plates; posterior cervical systems; posterior lumbar systems; anterior lumbar systems such as standalone ALIF and ALIF plates; lateral plates; buttress plates; ISP plates; percutaneous screw systems; and other procedures.

### Anchor Extension Attachment Assemblies

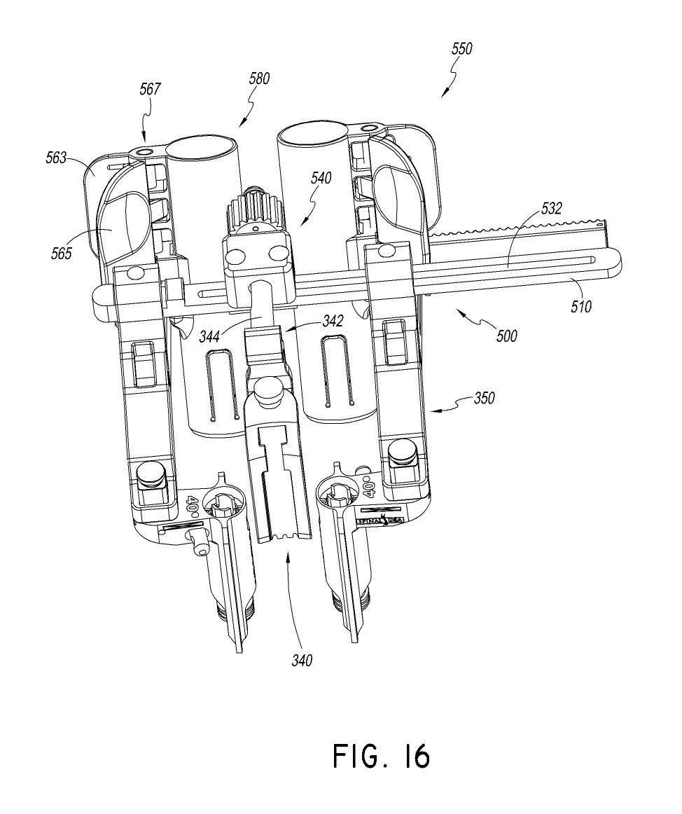

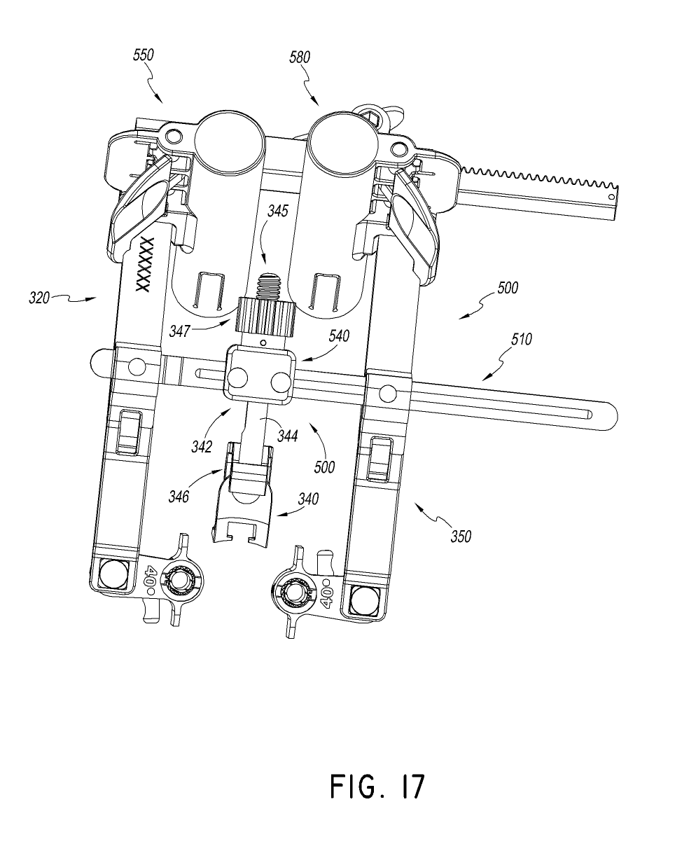

FIGS. 1 and 15-17 illustrate one embodiment of an anchor extension attachment assembly 550, which can be used to attach different anchor extensions to a retractor system. The assembly can include a tube assembly 580 having an arm attachment portion 560. The arm attachment portion can have at least one opening 564 that can be configured to fit around an arm of a retractor system 310. The arm attachment portion 560 can also include an arm attachment body 562 with a first flange portion 563, a second flange portion 565 coupled to the first flange portion in a spring-biased hinge arrangement 567. In the illustrated embodiment, the first flange portion is coupled to a hinge portion positioned between a tube portion of the tube assembly 580. The first flange portion is also coupled to the arm attachment body 562. In some embodiments, the first flange portion is fixed relative to the hinge portion and the arm attachment body 562. The second flange portion is movably coupled to the hinge portion. The hinge portion comprises one or more springs to bias the second flange portion away from the first flange portion and into engagement with an opening in an arm of the retractor system. In some embodiments, the second flange portion has a hook portion 569 extending from the body of the second flange portion to engage with an edge of the opening in the retractor arm in a locked orientation. In some embodiments, the spring or other biasing means can supply force such that the anchor extension attachment assembly resists inadvertent medial-lateral motion, but can be overcome by the user to cause purposeful medial-lateral motion.

In the illustrated embodiment, when the second flange portion is biased away from the first flange portion, the hook portion engages an edge of a slot in the arm and acts to couple the anchor extension attachment assembly to the arm of the retractor and resist movement of the anchor extension attachment assembly relative to the retractor arm. When movement of the anchor extension attachment assembly relative to the retractor is desired, a user can depress the second flange portion toward the first flange portion against the spring biasing force to rotate the second flange portion toward the first flange portion. Rotation of the second flange portion toward the first flange portion causes the hook portion to move out of engagement with the retractor arm and allows the anchor extension attachment assembly 550 to be moved relative to the retractor arm or removed from the retractor arm. When the anchor extension attachment assembly 550 is moved to a desired position, the user can release the pressure against the second flange portion and the spring force of the hinge portion will cause the second flange portion to engage the retractor arm and hold the anchor extension attachment assembly 550 in position relative to the retractor arm.

Other arrangements for engaging and disengaging the anchor extension attachment assembly 550 to the retractor arm are also possible. For example, a suitable flange and hinge arrangement to quickly and easily engage and disengage the anchor extension attachment assembly 550 to the retractor arm can include other mechanical engagements with the retractor arm.

In some embodiments, the tube assembly 580 can be configured to couple to an anchor extension that passes through the tube assembly. In some embodiments, the tube assembly 580 can be configured to attach to and tighten about an anchor extension that passes through the tube assembly. In some embodiments, the anchor extension passes through the tube assembly and can be oriented at varying angles.

### Medial Blade Assembly

FIGS. 1 and 15-17 illustrate one embodiment of a medial blade assembly 500. A medial blade assembly 500 can be attached to the retractor body 320, such as to one or more of the arms 350. The medial blade 340 can be configured to move in a medial-lateral direction when placed within a patient, helping to retract tissue and create a clear view of the vertebrae. The medial blade assembly can include an assembly attachment portion 510, a medial blade positioning portion 540, and a medial blade arm 342 that can attach to a medial blade 340.

In some embodiments, the medial blade assembly can attach to the arms through attachment openings 354 on the arms. As shown, in some embodiments, the attachment openings 354 are defined in portions of the retractor arm extending beyond a pivot location coupling one arm portion to another arm portion. The attachment openings 354 are configured to receive an arm connecting section 520 of the medial blade attachment assembly 510. The arm connecting section 520 can be configured to attach the medial blade assembly to one or more arms of a retractor system. In some embodiments, a medial blade assembly 500 can be configured to connect to an arm 350 via one or more connecting pins which can be positioned within one or more corresponding holes in an arm of the retractor system. In some embodiments, a medial blade assembly 500 can be configured to connect to an arm 350 without connecting pins and/or holes in an arm of the retractor system. The arm connecting section 520 can be adjustably connected to the retractor arms and/or a medial blade positioning section 540. For example, in some embodiments the arm connecting section 520 can have one or more grooves or notches 532 that can be positioned to receive one or more projections of the retractor arms and/or the medial blade positioning section 540 or pins or screws attached to the medial blade positioning section. This can allow the positioning section 540 to move relative to the retractor arms and/or the arm connecting section 520 along an axis of the grooves or notches.

A blade arm 342 can be attached to the medial blade positioning section 540. In some embodiments, the blade arm and positioning section can be integrally formed. In some embodiments, the blade arm and positioning section can cooperate to move relative to each other to move the medial blade. In some embodiments, an adjustment section 344 of the blade arm can be slideably positioned within an opening in the positioning section 540. In some embodiments an adjustment section 344 of the blade arm can comprise a threaded portion 345 configured to be coupled to an adjustment knob 347 positioned on an opposite side of the positioning section 540 and configured to control movement of the adjustment section 344 of the blade arm.

In some embodiments, the blade arm can be moved by sliding it along a longitudinal axis of the adjustment section through the opening in the positioning section 540. In some embodiments, turning the knob can engage threads on the adjustment section to cause the adjustment section to slide along its longitudinal axis. In some embodiments, the blade arm can be adjusted along two dimensions: moving the adjustment section 344 through the positioning section 540 along the longitudinal axis of the adjustment section, and moving the positioning section 540 and blade arm 342 along the longitudinal axis of the grooves or notches 532. Once the blade arm has been adjusted to a desired position, the knob in a stationary position can hold the position of the blade arm 342. In some other embodiments, a pin or screw can be tightened to lock the arm into the desired position.

The blade arm can also include a tool attachment section 346, to which tools can be attached before or after the blade arm has been moved into a desired position. The tool attachment section can have a variety of holes having a variety of configurations allowing the holes to receive a variety of tools. The tool attachment section 346 can additionally or alternatively have one or more blade attachment holes that can each be configured to receive a medial retractor blade.

As illustrated, in some embodiments the medial blade positioning section 540 can have a cutout 542 sized and configured to receive a blade arm 342. In some embodiments, blade arm 342 can have different sizes or configurations depending upon an expected desired positioning of the tool attachment section 346. For example, in some embodiments the blade arm can have one or more bends 343 which can affect the vertical positioning of the tool attachment section. In some embodiments, a bend can be used to lower the tool attachment section relative to the adjustment section 344. In some embodiments, a bend can be used to elevate the tool attachment section relative to the adjustment section.

### Medial Blades and Lighting Tools

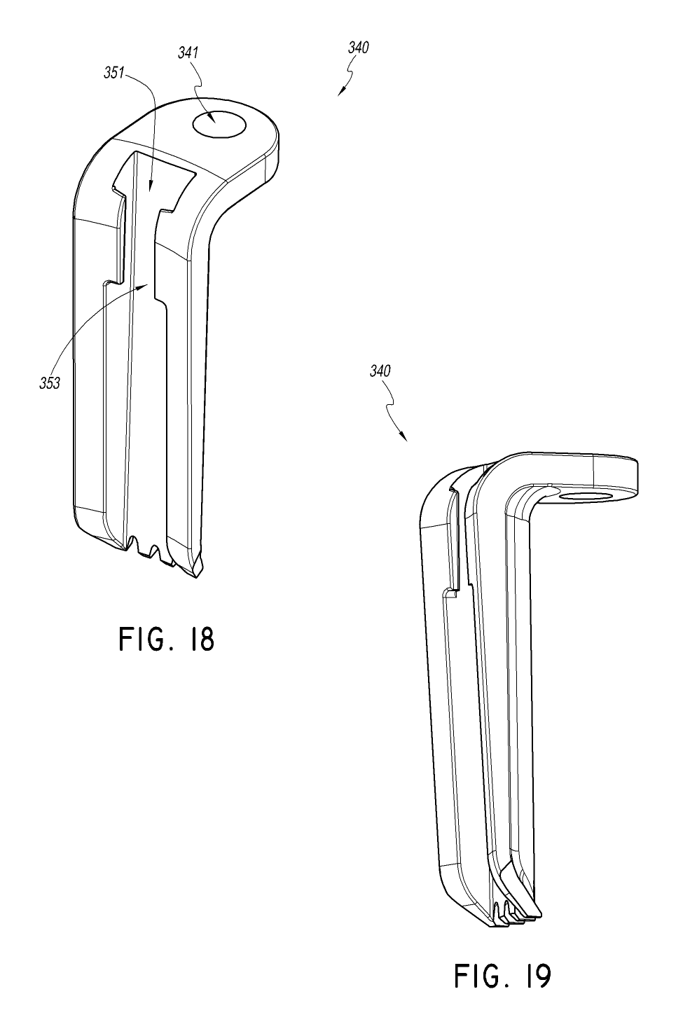

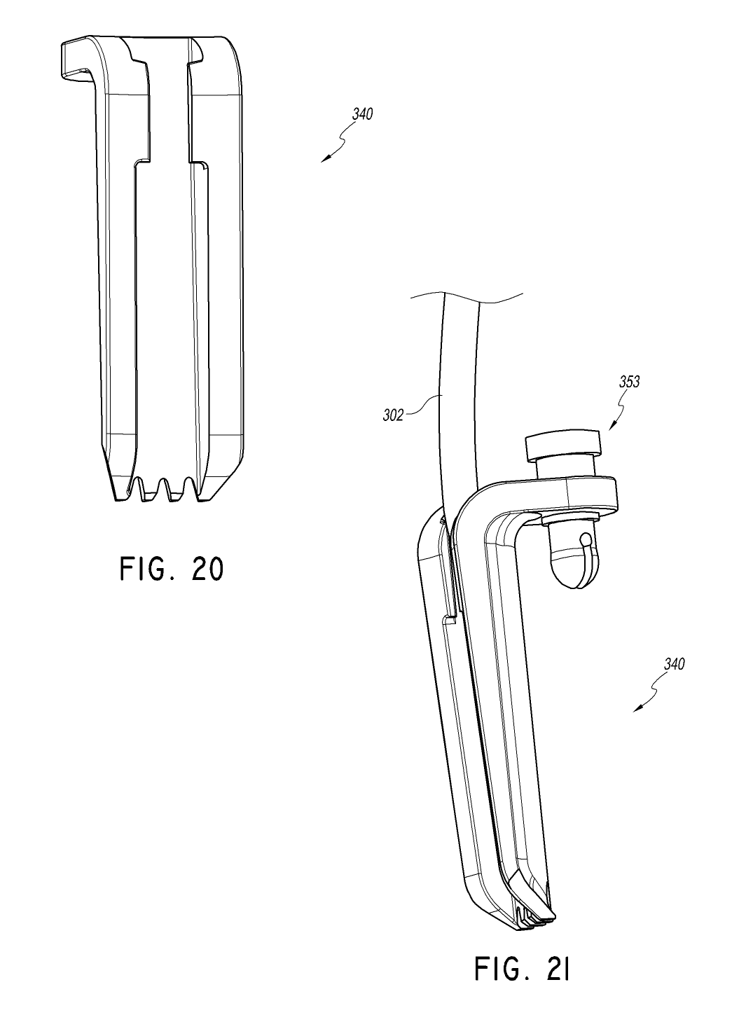

FIGS. 18-20 illustrate one embodiment of a medial blade 340. The medial blade has an opening 341 to receive a pin or other top-loading self-retaining attachment feature 355 for connection to the tool attachment section 346 such that an upper portion of the medial blade is positioned above the tool attachment section 346 when assembled. The medial blade has a recess or slot 351 that extends the length of the medial blade. The medial blade has tabs 353 configured to hold a lighting element 302, such as, for example, a malleable light wand, within the recess. The medial blade has a thicker proximal portion that tapers to a thinner distal portion. The thicker proximal portion in some embodiments has a thickness of about 3 to 4 mm. The thinner distal portion in some embodiments has a thickness of about 2 mm. The thicker proximal portion limits deflection of the medial blade in some embodiments.

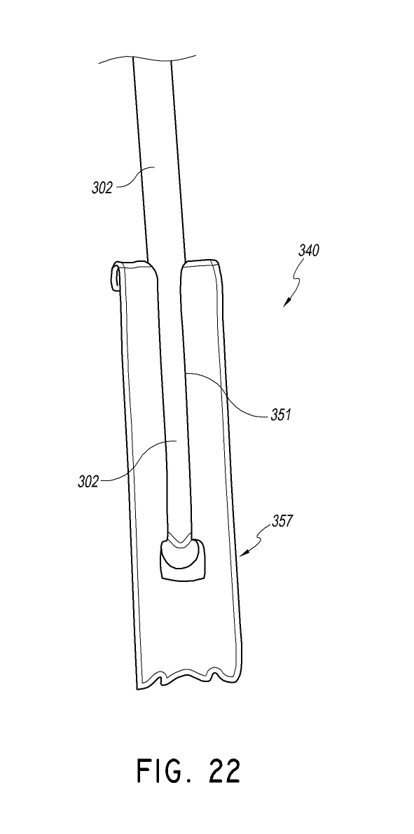

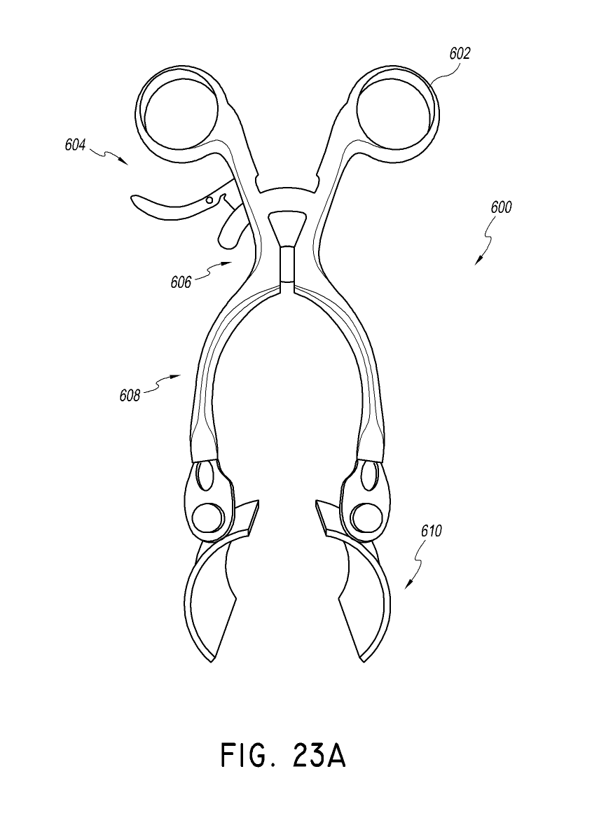

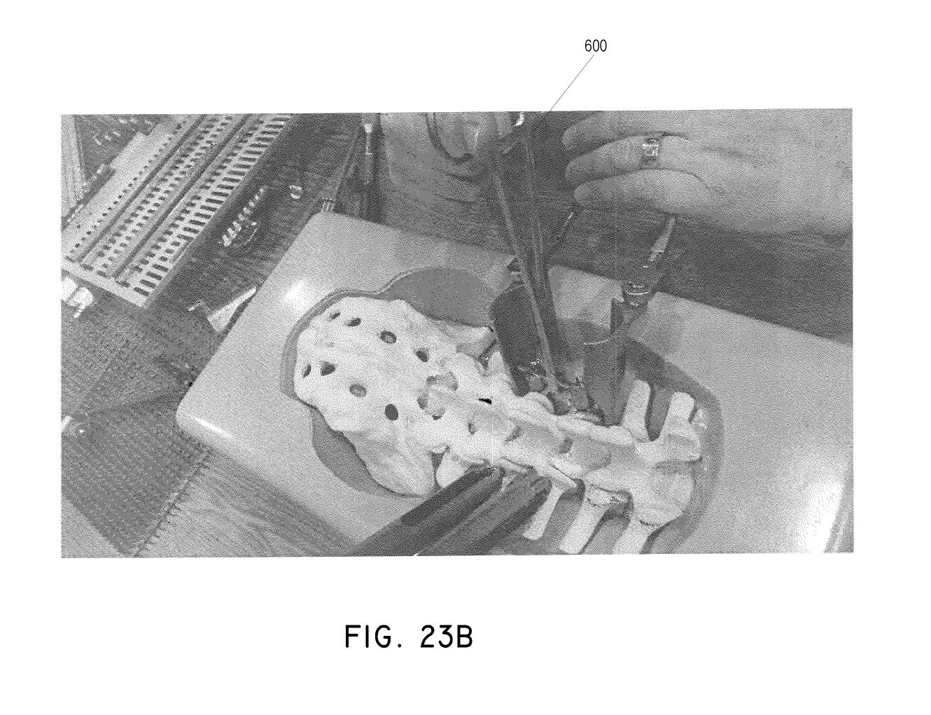

FIGS. 21-22 illustrate another embodiment of a medial blade and a lighting element. The medial blade is similar to the medial blade of FIGS. 18-20 except as shown and described below. For example, the medial blade of FIGS. 21-22 has a recess or slot that extends partially along the length of the medial blade and includes a widened portion 357 of the recess to allow for light to exit the recess. In some embodiments, the slot ends approximately 20 mm from the distal tip.

### Spacing Tool

FIGS. 23A-B illustrate one embodiment of a minimally invasive spacing tool 600 that can be used as described with respect to various embodiments described herein. For example, the tool has first and second handle portions 602, a ratchet mechanism 604, a pivot portion 606, first and second arms 608, and first and second spacer portions 610. The tool can be used to provide access to a surgical site. For example, when retractor blades are removed from screw heads, the tool can be used to provide space for rod insertion. As the handle portions are actuated, the spacer portions can be widened. The ratcheting mechanism holds the spacer portions in the widened configuration while the procedure is performed.

### Minimally Invasive Tower Access Device

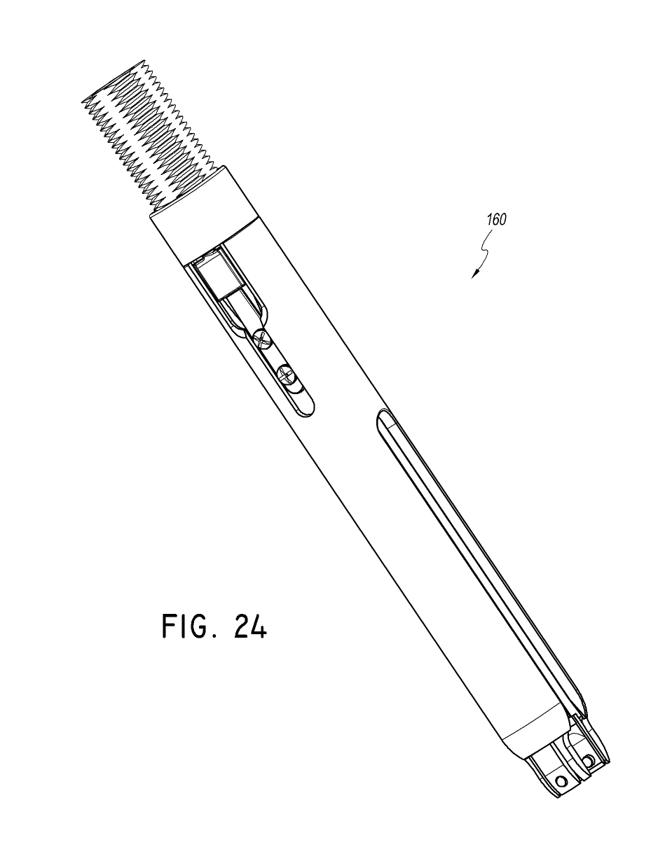

FIG. 24 illustrates one embodiment of a minimally invasive tower access device 160 that can be used as described with respect to various embodiments described herein. For example, the tower 160 can be used to deliver a spinal screw to a location proximate to a bone member where the spinal screw can be inserted. A tower can also serve as a portal or opening extending from the bone member to outside of the patient, through which instruments and implants (such as rods) can be delivered. In some embodiments, towers can be used to attach pedicle screws to the pedicles of vertebrae adjacent an intervertebral space to be operated on. Towers are described in more detail in U.S. Provisional Patent Application No. 61/653,853, filed on May 31, 2012, and U.S. Patent Application Publication No. 2012/0022594 A1, published Jan. 26, 2012, both of which are hereby incorporated by reference in their entireties and are to be considered a part of this specification.

### Retractor

FIGS. 1, 15-17, and 28 illustrate one embodiment of a retractor body 320 that can be used with a MAS retractor system. As described above, the arms 350 can be attached to a variety of anchor extensions, such as lateral blade assemblies and MIS towers. Once the arms 350 are attached to the desired anchor extensions, the arms and anchor extensions can be moved apart, which will tend to distract the disc space about which the extensions are attached. The arms can move according to various embodiments. As illustrated, the retractor body 320 can include a cross bar 322 with ridges or teeth 327. Both arms can be attached to the cross bar. In some embodiments, one arm can be attached with a moveable latch mechanism 480, which can have a latch 482 that can be spring-biased to lockingly engage the notches in the cross bar. Pushing on the latch can release it, allowing the arms to be moved closer together or farther apart. In some embodiments, the arms can be manually moved closer together or farther apart. In some embodiments, an adjustment screw 484 can be turned to help move the arm attached to the latch mechanism 480 along the cross bar 322.

The arms 350 can include a collar 356 that is sized to fit over the post 420 of a blade assembly 330. Preferably, the collar is oriented such that when a blade assembly is positioned within the collar, the blade plane (described above) is either generally parallel to or generally perpendicular to the cranial-caudal line of the patient or the cross bar 322 of the retractor body 320.

In some embodiments, the arms 350 can comprise a plurality of sections joined by articulating joints, and the most lateral sections can be configured to attach to the lateral blade assembly. In some embodiments, the arms 350 have a first section 472 and a second section 474, with the first section configured to connect to a lateral blade assembly. As illustrated, the first section 472 can articulate relative to the second section. In some embodiments, articulation can be locked, such as through the use of a screw, locking pin, or other device to hold the sections of the arms at a desired angle. The articulating arms allow the collar 356 to fit over the post 420 of a blade assembly 330 when the blade assembly has been angled away from the spine (such as when the surgeon desires to perform a TLIF) while still allowing the cross bar 322 to remain flat against the patient. This can minimize interference with or undesired forces on any anchor extensions attached to the retractor on the contralateral side and/or to the second section 474 of the arms 350.

In some embodiments, as described above, the retractor body can be configured to connect to a medial blade assembly 500 (such as that visible in FIG. 1) that can position a medial blade 340 between lateral blade assemblies 330. As discussed above, the medial blade can be configured to move medially, riding up the spinous process, to retract tissue and open a visual and operational space for the procedure. The medial blade assembly preferably attaches to the first section 472 of the arms 350, such that it can be positioned at the same angle as the lateral blade assemblies 330. Having all of the blades aligned at the same angle can help minimize damage to the multifidus muscle when the disc space is accessed. In some embodiments, particularly when a TLIF is the desired procedure, the blades can be positioned at an angle between 25 degrees (or about 25 degrees) and 30 degrees (or about 30 degrees). Other angles and approaches are considered. In some embodiments, as described above, the medial blade assembly 500 can be configured to allow a medial blade 340 to rotate independently of the angle of the arms 350.

In some embodiments, a retractor body can have a second section 474 of the retractor arms 350 that can rotate relative to the cross bar 322 in addition to being able to rotate relative to the first section 472. Screws or pins can be used to tighten and/or lock one or more of the sections of the arms 350 relative to adjacent sections.

### Minimally Invasive Retractor Systems for Extended Tab Percutaneous Screws

FIGS. 25-27 illustrate another embodiment of a retractor system 700 for use in a minimally invasive surgical system. The retractor system 700 can be configured for use with extended tab percutaneous screws 702.

In some embodiments a surgeon may attach four extension attachment assemblies 704 to the arms of the device. This could allow, for example, the surgeon to attach the retractor body to extended tab percutaneous screws on an ipsilateral or operational side of a patient's spine and to towers on a contralateral side of the patient's spine. In some embodiments the retractor body is attached to extended tab percutaneous screws on either or both sides of the patient's spine. Spring-loaded tabs and/or levers 706 can engage arms of the retractor assembly to provide for adjustment in the positioning of the extension attachment assemblies along the retractor arms. As used herein, the term extended tab screw is a broad term and is used in its ordinary sense, and in some embodiments can refer to a screw that preferably extends beyond a patient's skin when the screw has been attached to a patient's vertebra. It is understood that the extended tab can refer to either integral features of the screw housing or removable features that serve the same purposes.

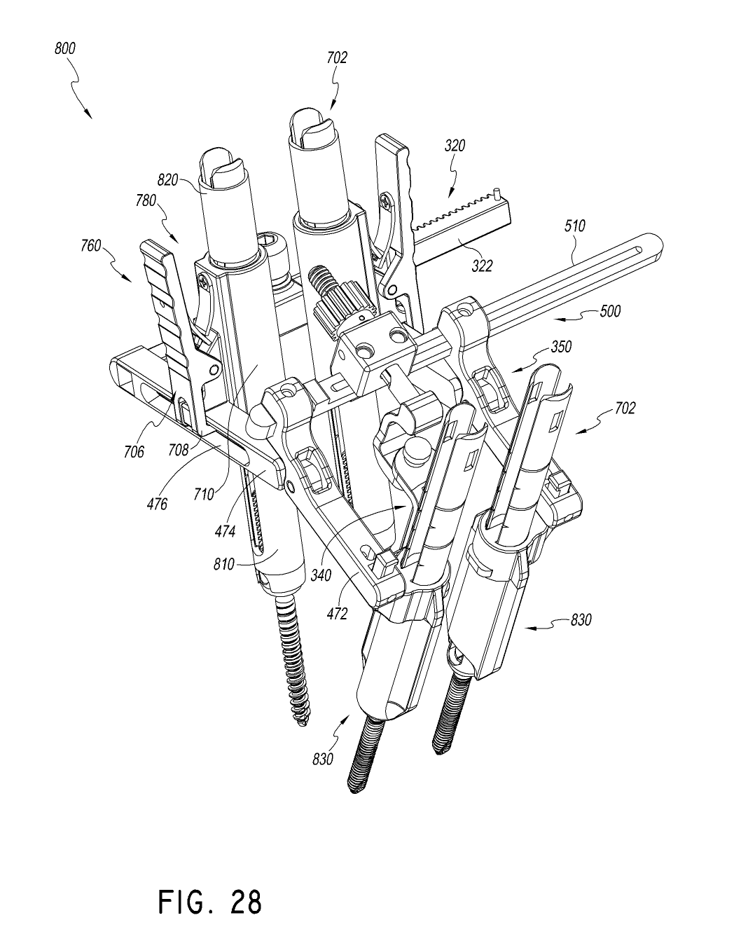

FIG. 28 illustrates an alternate embodiment of a retractor system 800 for use in the minimally invasive surgical system and that includes extended tab percutaneous screws 702. The system can include the use of extended tab percutaneous screws on both an ipsilateral or operational and on a contralateral side of the patient's spine, although in various embodiments one or more screws on one or more of the operational and contralateral sides can use standard length screws and instrumentation as described above.

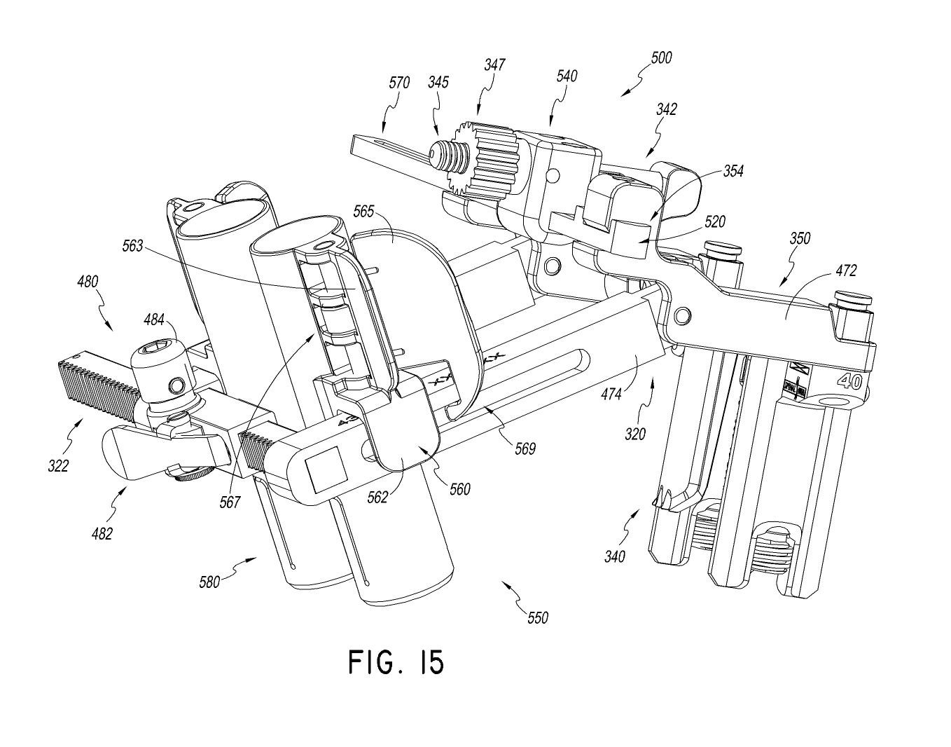

Many aspects of the retractor system 800 can be the same as or similar to aspects illustrated and/or described above. Unless otherwise noted, similarly numbered components will operate the same as or similar to those previously described. Thus, for example, in some embodiments a retractor system 800 can include a medial blade assembly 500 that can operate the same as that described with respect to FIGS. 15 through 17. Similarly, the retractor assembly can include a retractor body 320 with one or more retractor arms 350, such as retractor arms that include a first section 472 that can articulate relative to a second section 474.

In some embodiments, the retractor body can attach to extended tab screws 702 via attachment assemblies, such as a tube assembly 780 that can be used on a contralateral side and/or a lateral blade assembly 830 that can be used on an operational side. It is understood that various different types of attachment assemblies can be used and that the illustrated attachment assemblies can be used on different sides of the spine. For example, in some embodiments the operational side can have one or more tube assemblies 780 that can be used to attach the screws on the operational side to the retractor body 320. In some embodiments, for example as illustrated, the tube assembly 780 can include an arm attachment portion 760 and a tubular portion 710 configured to be positioned around the extended tab screw 702.

In some embodiments, one or more sleeves can be positioned between an attachment assembly, such as the tube assembly 780, and an extended tab screw 702. For example, in some embodiments, a first sleeve 810 and a second sleeve or cap 820 can be positioned around a screw and the tubular assembly can slide over one or both of the sleeves. As described in more detail below, this can help allow for distracting disc space without accidentally breaking the extended tabs of the screw.

In some embodiments, the tube assembly 780 can include a tab or lever 706 with a hooked or projecting portion 708 that can be biased to move toward the tube 710. The projection 708 can fit within a groove or slot 476 on a portion of an arm 350, thereby attaching the tube assembly to the retractor body 320. In some embodiments, the tab or lever 706 can extend in a direction generally parallel to a longitudinal axis of the tube assembly. Pressing against the tab or lever can pivot the hook or projection 708 away from the tube 710 and out of the slot 476, thereby releasing the tube assembly and allowing for its removal. Having the tab or lever oriented in this way can make it easier for a surgeon to access and press the tab or lever to release the tube assembly. The tab can be pressed with the surgeon's hands and arms at an angle that minimizes interference with access to the surgical site.

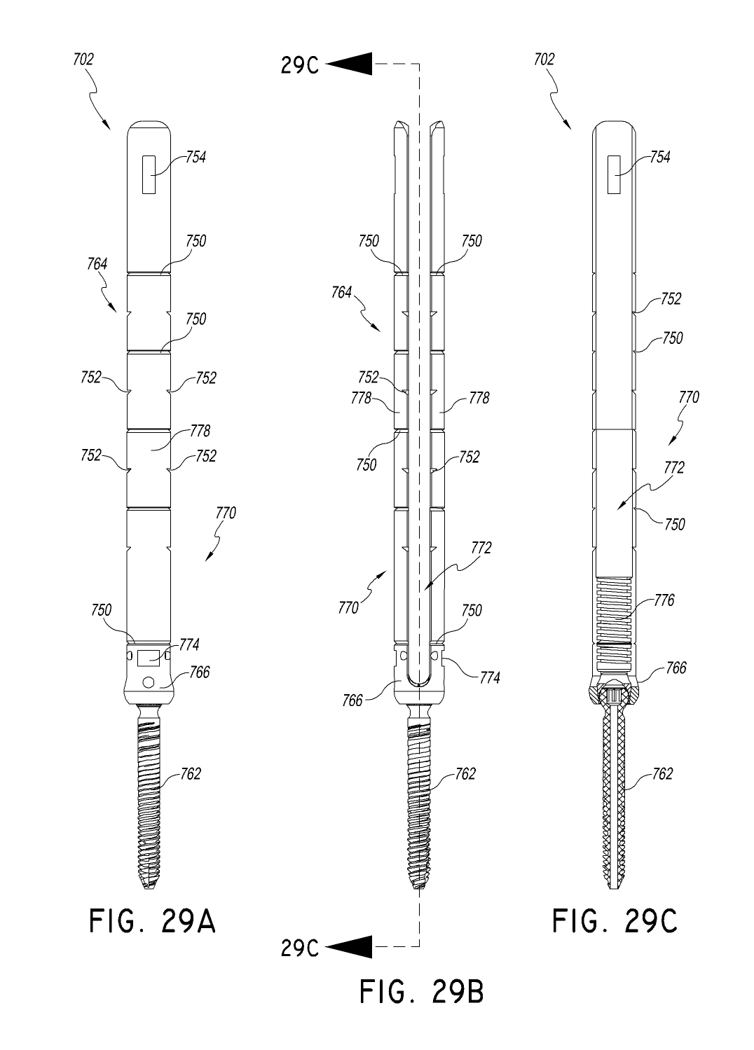

FIGS. 29A through 29C illustrate one embodiment of an extended tab screw 702. FIGS. 29A and 29B illustrate side views of the screw and FIG. 29C shows a cross-sectional view. The screw can include a shank or shaft 762 that is preferably cannulated and includes external threading. The screw can also include a housing 770, which can include a base portion 766 and extended portion 764. The base portion 766 and extended portion 764 together may form a monolithic housing. Preferably, the extended portion and the base portion include at least two walls, prongs or tabs 778 that define an opening or transverse channel 772 between them. The extended portion in various embodiments can be formed of different materials. In some embodiments, it can be generally rigid and can be formed, for example, of a hard metal or plastic. In some embodiments, it can be flexible. In some embodiments, the channel 772 can be used to receive a fusion rod and/or help guide a fusion rod to the base of the channel in the base portion 766. In some embodiments, the structure of the base portion 766 and the shaft 762 are identical to the shaft 76 and housing 70 illustrated and described with respect to FIG. 2, further details of which are provided in U.S. Patent Application Publication No. 2010/0241175, the entirety of which is hereby incorporated by reference. The base portion 766 and shaft 762 can rotate polyaxially relative to each other, but during insertion the screw driver utilized may immobilize the base portion 766 relative to the shaft 762 to create a temporary monoaxial screw.

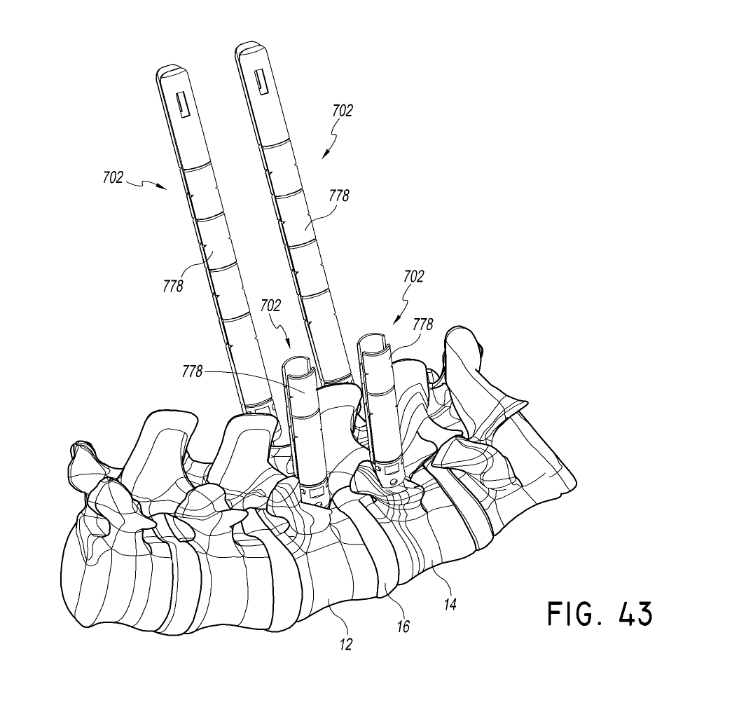

In various embodiments, the extended portion 764 can include a variety of features that can assist with surgical procedures using the screw 702. For example, in some embodiments, the extended portion can include one or more frangible or weakened sections 750. In some embodiments, these sections can be score marks, areas of thinner material, or have other structural features such that the extended portion can break more easily at the weakened sections than at other sections of the extended portion 764. This can help with removing sections of the screw as needed during a surgical procedure, described in more detail below. In some embodiments, the lower most or distal most weakened section 750 can mark the boundary between the base portion 766 and the extended portion 764, such that the entirety of the extended portion 764 can be removed from the base portion if desired. In some embodiments, the extended tab screws on the operational side of the patient may be different from the extended tab screws on the contralateral side. For example, on the contralateral side the extended tab screws may have only one frangible or weakened section 750 marking the boundary between the base portion 766 and the extended tab section 764, as in some procedures it may only be desired to break off the extended tab portion 764 at this location. On the operational side, the extended tab screws may have a plurality of frangible or weakened sections 750 as shown in FIGS. 29A to 29C. This plurality of frangible or weakened sections 750 provide for a number of locations where an upper portion of the extended tab portion 764 may be broken off, so that the remaining extended tab portion has a height at about the level of the patient's skin (as shown in FIGS. 42 and 43 described below). For example, these frangible or weakened sections 750 may be placed 20 mm apart.

In some embodiments, the screw 702 can also include one or more locking notches 752 positioned longitudinally along the screw. In some embodiments, the notches can extend perpendicular or generally perpendicular to the longitudinal axis of the screw. In some embodiments, for example as illustrated, the notches can traverse the channel 772.

The notches 752 can be used to help lock an attachment assembly, such as the lateral blade assembly 830, into a desired position along the screw 702. Examples are described further below. In some embodiments, each of the locking notches 752 can be generally the same size, shape, and/or depth. In some embodiments, the notches can have different shapes, sizes and/or depths. In some embodiments, each notch can have a different size, shape, and/or depth that corresponds to a particular size of attachment assembly, such as described further below.

In some embodiments, such as shown in FIGS. 29B and 29C, an extended tab screw 702 can include one or more openings 754 on a proximal end. Preferably, each tab 778 includes an opening 754 aligned with the opening 754 of an opposite tab. In some embodiments, the screw housing 770 can include a portion with internal threading 776. In some embodiments, the internal threading 776 can be entirely within the base portion 766. In some embodiments, the internal threading can extend from the base portion into the extended portion 764. The internal threading can be used to receive a component, such as a set screw 66 (e.g., FIG. 2) that can help maintain a fusion rod into position.

In some embodiments, a screw 702 can also include one or more attachment portions 774, preferably in the base portion 766. In some embodiments the attachment portions can include a recess, for example as illustrated. In some embodiments, the attachment portions can include a projection or other component. The attachment portions can be used to attach the screw to other components of a retractor system, such as an attachment assembly, sleeve, or tower.

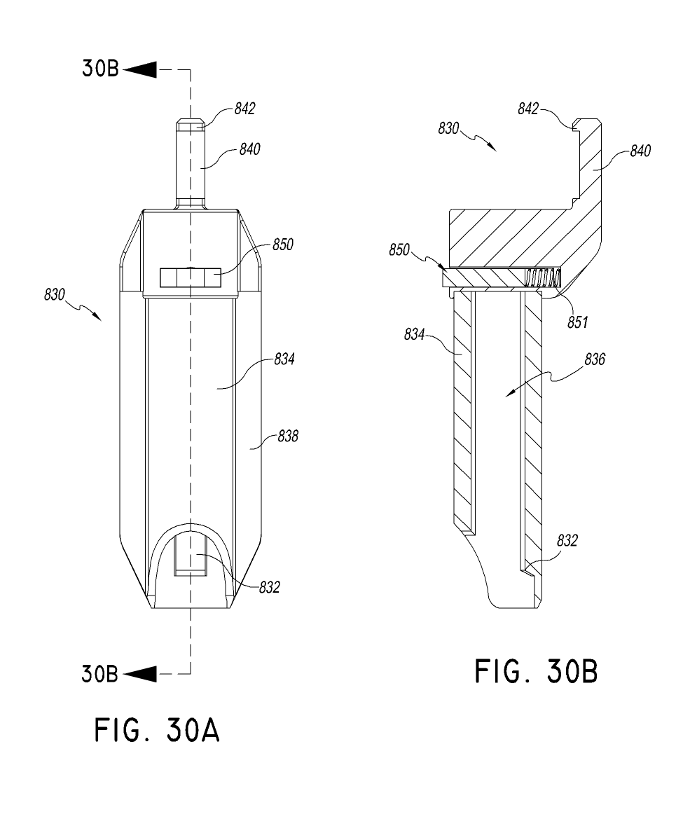

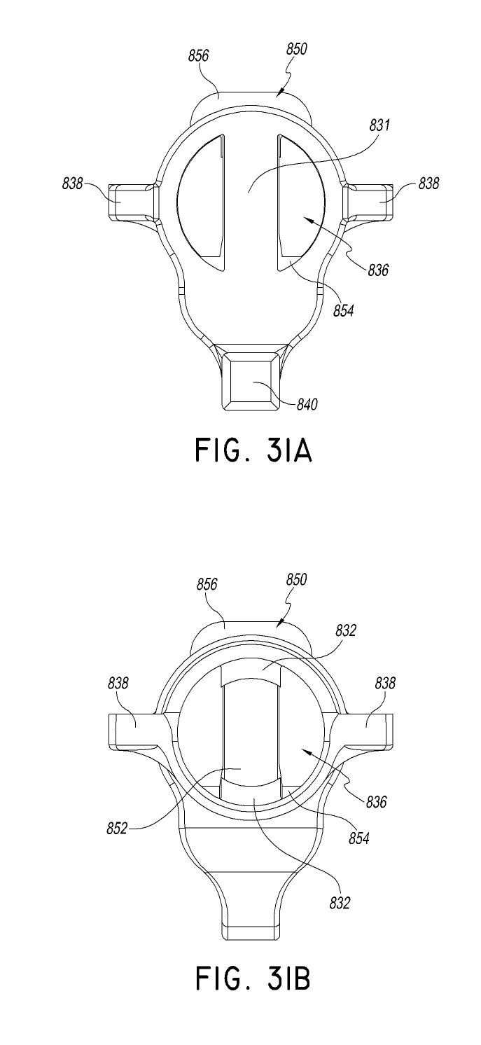

FIGS. 30A through 31B illustrate one embodiment of a lateral blade assembly 830 that can be used with a retractor system, such as the retractor system 800. In some embodiments, the lateral blade assembly 830 is utilized with the extended tab screws 702 on the operational side of a patient. FIG. 30A is a front view of the blade assembly 830 and FIG. 30B is a cross-section. FIGS. 31A and 31B are top and bottom views, respectively. Similar to various embodiments described above, the lateral blade assembly can include a central section 834 and one or more wing or blade extensions 838 extending from the central section. These can help provide for additional tissue retraction.

The blade assembly can also include a post 840 that can be used to help attach the blade assembly to an arm 350 of the retractor body 320 (as shown for example in FIG. 28). In some embodiments, the post 840 can include a projection 842 on at least one side of the post. This can help prevent the post from undesirably moving out of connection with the corresponding retractor arm, as described further below.

In some embodiments, the lateral blade assembly can include at least one interior projection 832 that can be configured to fit between opposing tabs of an extended tab screw when the blade assembly is positioned around the screw. This can help the tabs retain their position and prevent them from flexing toward each other. Preferably, projection 832 can be configured to fit at a most distal end of the opening or gap 772 between tabs or prongs 778. Preferably, at least a portion of the central section 834 can extend distal to any gap or opening in the screw housing 770 and can be positioned against a distal end of the base portion 766. If the retractor body is used to apply a force on the screws through the lateral blade assemblies, such a distraction force is thus preferably not received entirely on the tabs 778. This can help prevent any distracting force from breaking the tabs (such as at the frangible portion 750) when not desired.

FIG. 31A illustrates a top view of the blade assembly 830 and FIG. 31B illustrates a bottom view of the blade assembly. In some embodiments, the blade assembly 830 can have a central bore 836. The tabs 778 of a screw can fit within the bore. In some embodiments, a locking button 850 is provided that includes a central waist 852 that can pass across a portion of the central bore and that can fit within the gap between tabs 778. In some embodiments, as shown in FIG. 31A, the blade assembly can include a section 831 that extends across the central bore 836 and that can be generally aligned with the waist 852 of the button. This section 831 can protect the button from accidental damage caused by any instrumentation being used during a procedure.

FIGS. 31A and 31B also illustrate a portion of the locking end 854 of the button 850 that extends into the central bore 836 when the button is in a locked position, as illustrated. In some embodiments, the locking end 854 in the locked position can be configured to engage a screw 702 to prevent the blade assembly 830 from moving longitudinally relative to the screw. In some embodiments, the locking end can fit within locking notches 752 (such as notches described with respect to FIGS. 29A through 29C) of tabs 778. In some embodiments, the button in the locked position may also prevent insertion of a screw through the blade assembly 830. In some embodiments, the button may have an angled surface, described below, that can interact with a screw being inserted and move the button into an unlocked position.

When the button 850 is in the unlocked position, the locking end 854 preferably does not engage the screw 702, thereby allowing the screw to move longitudinally relative to the blade assembly 830. In some embodiments, the button in an unlocked position does not extend into the central bore 836 of the assembly. In some embodiments, when the button is in an unlocked position a portion of the locking end may still extend into the central bore. The button can preferably be manually moved into the unlocked position, such as by pushing an actuation end 856 that can extend outward from the blade assembly. In some embodiments, the button can be biased into the locked position. For example, as shown in FIG. 30B, in some embodiments a spring 851 can bias the button into the locked position.

In some embodiments, blade assemblies of different sizes can have buttons with locking ends 854 of varying shape, size, and/or width. In some embodiments, the locking end 854 of a particular blade assembly can be configured to fit and lock within only one set of notches 752 at a desired position on the percutaneous screw 702. In some embodiments, the notches 752 and locking ends 854 can be configured such that a locking end 854 of a particular blade assembly will not engage notches above a desired height. For example, in some embodiments the notches above the desired height may be too narrow to receive the locking end 854 (i.e., the locking end may be too thick to enter the notches). In some embodiments, locking notches may grow progressively larger (e.g., progressively wider so as to receive thicker aspects of the locking end) as they move down the screw housing. Preferably, blade assemblies are configured to lock with notches at a height such that when in use the blade assembly is lockable when a top of the blade assembly is generally level with a patient's skin.

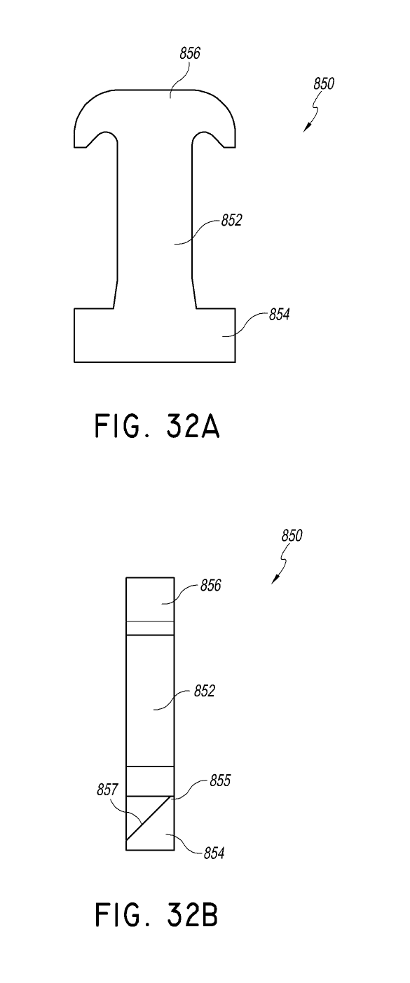

FIGS. 32A and 32B illustrate a top view and side view of the button 850. In some embodiments, the button can be formed as an integral piece. As shown in FIG. 32B, in some embodiments the locking end 854 of the button can include a locking edge 855 that can be configured to fit within a notch or notches 752 of a screw 702. FIG. 32B also illustrates an angled surface 857 that can interact with a screw to move the button into an unlocked position when the screw is inserted into the blade assembly 830. When the locking edge 855 is engaged with a notch, however, it preferably locks the blade assembly from being removed from the screw.

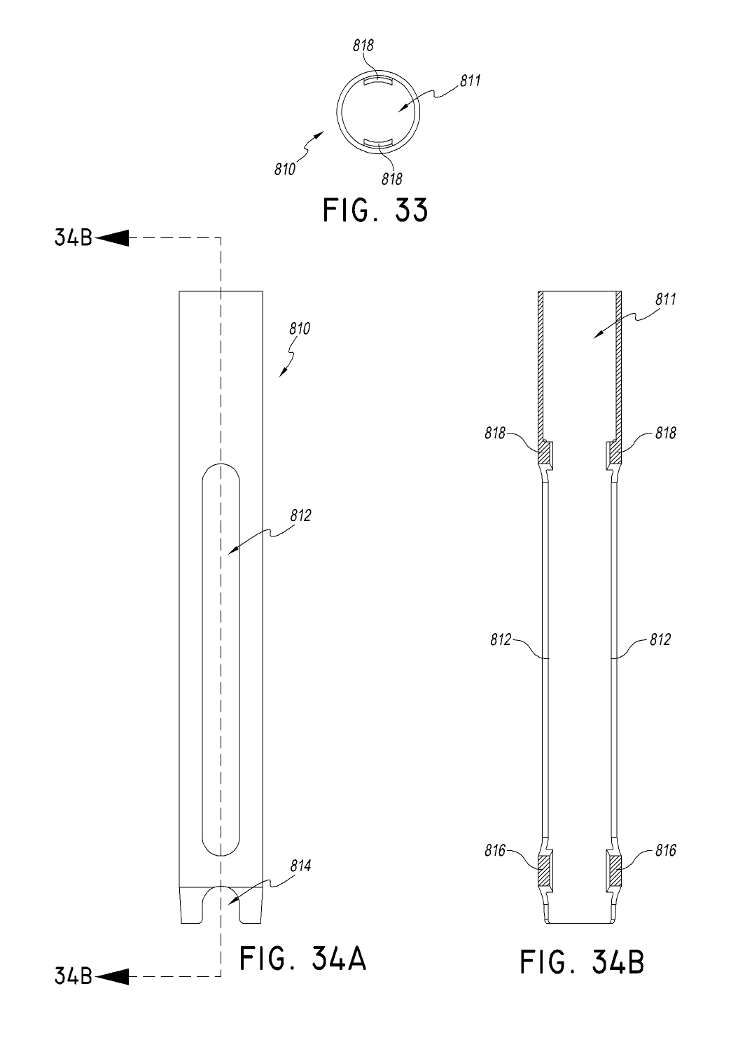

FIGS. 33 through 34B illustrate one embodiment of an attachment assembly that can be positioned over an extended tab screw such as on the contralateral side of a patient as shown in FIG. 28. FIG. 33 illustrates a top view of a first sleeve 810. FIG. 34A illustrates a side view and FIG. 34B illustrates a cross-sectional view of the sleeve. As shown in FIG. 33, in some embodiments the sleeve 810 can include a central longitudinal bore 811 that can receive the tabs 778 of the screw 702. The sleeve can also include one or more interior projections, such as projections 818, that can be configured to fit within a gap 772 between tabs of the screw. As described above, this can help prevent the tabs from moving toward each other during a procedure, thereby helping the screw retain a desired shape. In some embodiments, such as illustrated in FIG. 34B, the sleeve can include one or more distal interior projections 816 and one or more proximal projections interior 818. In some embodiments, the sleeve can include one or more projections that extend from a distal end of the sleeve to a proximal end of the sleeve.

In some embodiments, such as shown for example in FIG. 34A, the sleeve can include one or more transverse cut-outs. For example, in some embodiments, the sleeve can include a transverse cut-out 812 that can extend along a longitudinal portion of the sleeve. In some embodiments, the cut-out 812 can be large enough for a fusion rod to fit through it, and in some embodiments, it can be sized to be smaller than a diameter of a fusion rod configured for use with the screw 702. The longitudinal cut-out can help provide visualization during a procedure, allowing a surgeon to see through a portion of the sleeve. Preferable, such as illustrated, any interior projections of the sleeve are longitudinally aligned with the cut-out, such that for the sleeve to fit over the tabs of an extended tab screw the cut-outs will be aligned with any gaps between the tabs. This can allow for a clear line of sight through the sleeve and through the screw to an operational space.

In some embodiments, the sleeve 810 can include a cut-out 814 in its base that is closed at its proximal end and open at its distal end. Preferably, the cut-out 814 is sized to receive a fusion rod and can be used to help position a fusion rod into a base 766 of a screw. In some embodiments, the fusion rod can be positioned within the opening or gap 772 of the screw housing 770 and moved to the distal end of the gap in the base 766 of the screw (shown in FIG. 29A). In some embodiments, when the fusion rod has been initially inserted into the gap 772 of the screw, the first sleeve 810 can be positioned over the screw and moved distally. The cut-out 814 can receive the fusion rod such that distal movement of the sleeve can drive the fusion rod distally toward a desired position in the base 766. Once the fusion rod is in position the sleeve 810 can be removed.

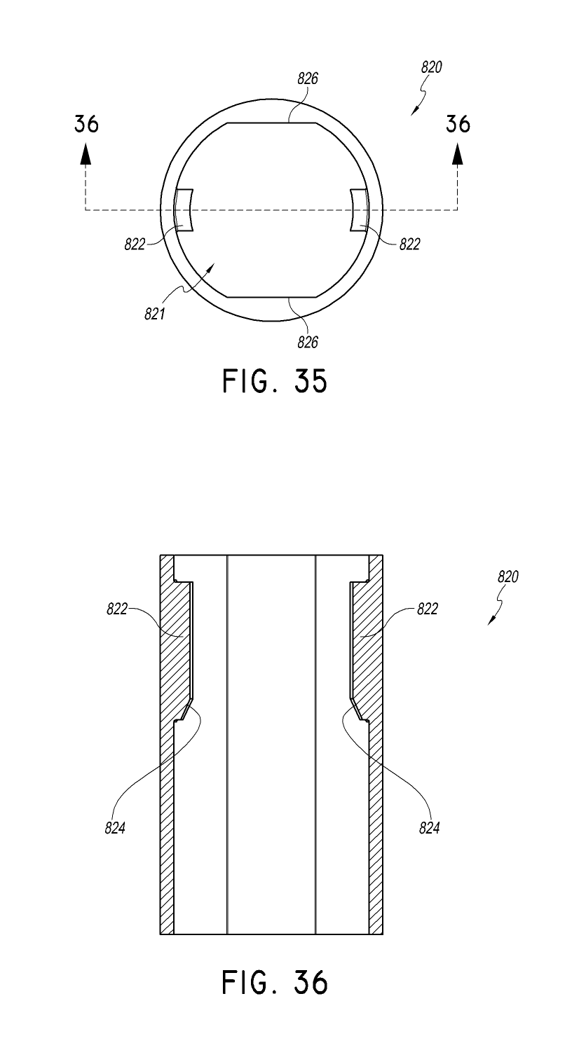

FIGS. 35 and 36 illustrate an embodiment of a second sleeve 820, another device that can be positioned around a screw 702 in some embodiments. FIG. 35 illustrates a top view of the second sleeve 820 and FIG. 36 illustrates a cross-section of the second sleeve. In some embodiments, the second sleeve can have internal projections 822 that can fit within opening 754 (FIG. 29B) to help retain the sleeve in position near a proximal end of the screw 702. As shown in FIG. 35, in some embodiments the sleeve 820 can have one or more flattened interior walls 826 along its bore 821 to ensure that the sleeve can only fit over the tabs 778 of a screw when the one or more projections 822 are aligned with the opening 754.

### Methods for Accessing Disc Space

Various embodiments of methods of using a retractor system to insert a spinal implant within a vertebral disc space are described. To begin, marks can be made on the positions on a patient's back that lie above both pedicles of the vertebrae on either side of the desired disc space. Using techniques known in the art, an incision is created on each marked spot. Either now, or later in the procedure, the surgeon can join the incisions on the same side of the spine. The surgeon can then use his or her finger to separate the muscle along the incision, preferably dissecting it along a single plane to make the healing process quicker and easier.

In some embodiments, a drill guide can be placed through the incision and onto the entrance to a pedicle. A drill can be advanced through the drill guide to drill a hole in the pedicle. A guide wire can then be inserted through the cannula of the drill guide and into the pedicle, and the drill guide can be removed. In some embodiments, the guide wire can be inserted through trocars, needles, or other hollow instruments instead of the drill guide.

The surgeon can select the desired bone screw and appropriate length lateral blade assemblies, such as lateral blade assemblies 30 or 330 described above with respect to FIG. 1 and FIG. 7. The surgeon can attach a screw extender 140 (e.g., FIG. 9A) to the blade assembly and then attach the blade assembly to a screw, as described above. Inserting the screwdriver 150 (e.g., FIG. 10) through the screw extender and into the blade assembly 30 can lock the screw extender into place, as discussed above, and the combination of the attached screw, blade, extender, and screwdriver can be inserted along the guide wire and into position on a pedicle on the operational side. In some embodiments, the screw can be inserted first and then the blade and screw extender can be inserted to attach the blade to the screw. In some embodiments, one or more dilators can be inserted prior to inserting the blade assemblies in order to expand a space for insertion of the blade assemblies. In some embodiments, the screw can be attached to other anchor extensions such as MIS tower assemblies or extended tab screw assemblies.

When the blade 30 is first inserted into the patient it is preferably oriented such that the blade plane (defined above) is parallel to the opening joining the incisions (i.e. generally parallel to the spine). Because the retractor blade shaft 100 (e.g., FIG. 5) can rotate independent of the retractor blade, as discussed above, if the blade assembly is attached to the screw after the assembly has been positioned within the patient, the retractor blade will be able to maintain its orientation relative to the patient.

Once the blade, screw, extender, and screwdriver are in place, the screwdriver can be used to drive the screw into the pedicle. Preferably, only the screw shaft and the screwdriver will rotate, and the remaining components will maintain their orientation relative to the patient. Once the screw has been fully inserted into the bone, the screwdriver can be removed and then the extender can be removed. The blade assemblies can be rotated 90 degrees (either before or after removing the blade extender), such that the blade plane is generally perpendicular to the spine. This will retract tissue and create a visual and operational space in which the surgeon can operate. The guide wire can be removed at any point after the screw has begun to enter the pedicle.

This procedure can then be repeated for the opposite pedicle on the operational side. Additionally, if desired by the operating surgeon, instrumentation, such as pedicle screws with towers, can be inserted into the opposite pedicles on the contralateral side either before or after inserting the lateral blade assemblies on the operational side. As discussed above, additional arrangements of anchor extensions can be used, such as using MIS towers instead of lateral blade assemblies.

Once all desired pedicle screws have been placed (e.g., two with blade assemblies on the operational side and two with towers on the contralateral side), the retractor body can be positioned over the patient's back and attached to the towers and lateral blades. If the surgeon desires to adjust the angle of the lateral blades, this can be done without affecting the positioning of the retractor body due to the articulated arms, as discussed above. In some embodiments, the surgeon can rotate the lateral blade assemblies by approximately 90 degrees to help establish an operational corridor. This can be done either before or after attaching the blades to the retractor body, or before or after adjusting the angle of the blades. In some embodiments, once an operational corridor has been established a surgeon may desire to improve access to an intervertebral space, such as by performing a facetectomy. The retractor body can distract the disc space by moving the arms of the retractor apart.

If desired, the surgeon can position a medial blade between the two lateral blades and attach the medial blade to the retractor. The medial blade can then be moved medially, retracting more tissue and broadening the visual and operational space. The standard TLIF, PLIF, TPLIF, or other procedures can then be performed. Following the procedure, the blades can be unscrewed from the pedicle screws in the same manner in which they were attached. If desired, rods can be inserted to join the pedicle screws, and the rods can be locked into place with set screws, as discussed above. Rods can also be inserted to join pedicle screws on the contralateral side, and the towers can be removed from the pedicle screws on that side.

If the surgeon desires to perform the procedure on multiple adjacent levels, then additional screws can be inserted in the next level using the same methods described above. This can be done before or after performing the methods described above. One advantage of this system is that the lateral blade assemblies can be symmetrical and can be used on either the cranial or caudal side of the operation (also referred to as the left or right side, from the surgeon's point of view). Thus, the blade or other anchor assembly adjacent the newly inserted pedicle screw on the operational side does not need to be removed and replaced, but can instead be rotated 180 degrees if necessary. The procedure can then proceed as described above.

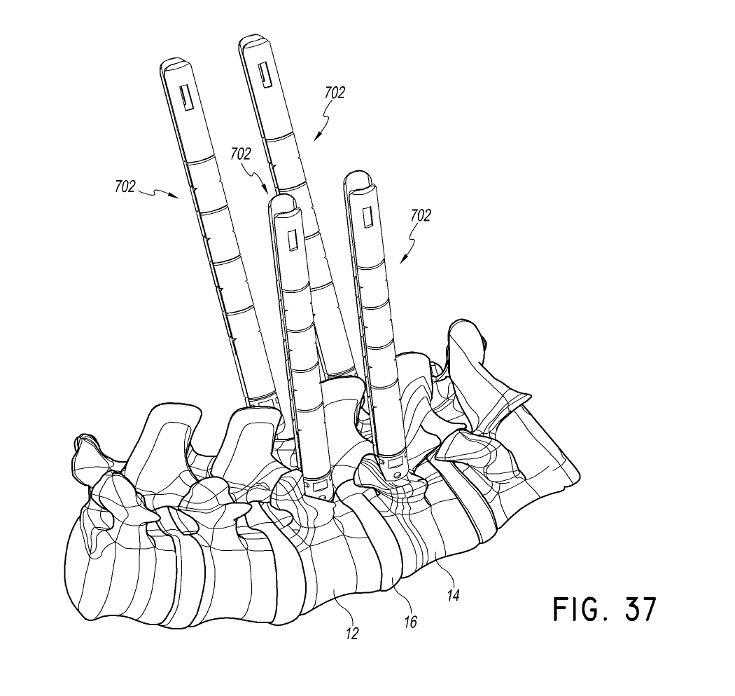

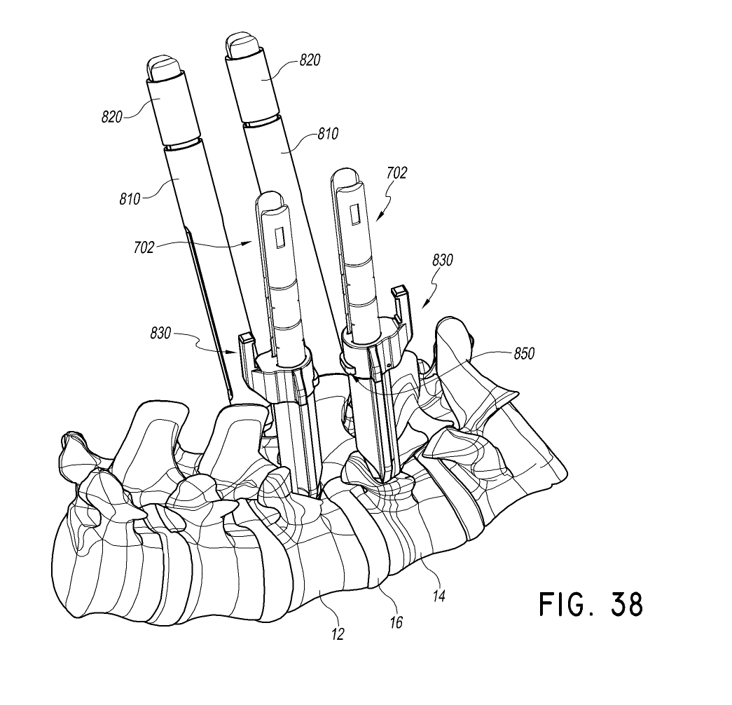

FIG. 37 through FIG. 43 illustrate one embodiment of a method of using a retractor system with extended tab screws to insert a spinal implant within a vertebral disc space. Unless described differently, it is understood that aspects of the methods described above can be used with a method of using a retractor system with extended tab screws. For example, it is understood that procedures using a retractor system with extended tab screws can be performed on multiple adjacent levels. Similarly, in some embodiments, the initial incisions and muscle separation techniques that are described above, as well as procedures for inserting a guide wire, can be used.

FIG. 37 illustrates a view of a section of a patient's spine with extended tab screws 702 inserted into the patient's spine. The screws can be inserted on an ipsilateral or operational side into a patient's spine, such as in the pedicles of a first vertebra 12 and a second vertebra 14 on opposite sides of a disc space 16. Screws can also be inserted into pedicles of the vertebrae 12, 14 on the contralateral side. In some embodiments, when multi-level procedures will be performed, screws can be inserted into a third vertebra or more vertebrae. In some embodiments, the screws may be inserted by first using a drill guide placed through an incision or incisions made into the patient and onto pedicles on the ipsilateral and contralateral sides, and drilling into the pedicles (for example to a depth of about 20 mm). Guide wires may be placed through the cannula of the drill guide and into the pedicles. The drill guide may be removed and the bone screws with extended tabs may be inserted over the guide wires and screws into the pedicles on the ipsilateral and contralateral sides. The guide wires may then be removed.

Once the screws have been positioned within the spine, instrumentation can be inserted over the screws, such as shown in FIG. 38. For example, on one side, such as the operational side, blade assemblies 830 such as those described above can be positioned over the screws 702. In some embodiments, blade assemblies can be inserted on both sides. In some embodiments, one or more sleeves, such as sleeve 810 and/or sleeve 820 can be inserted on the contralateral side. In some embodiments, one or more sleeves can be inserted on the contralateral and operational side. In some embodiments, instrumentation can be inserted over screws inserted on one side, such as the operational side, before any screws are inserted on a second side, such as the contralateral side.

Preferably, a blade assembly of a desired height is selected such that an upper surface of the blade assembly, such as the section 831 (FIG. 31A), will be at approximately the level of the skin. This can maximize tissue retraction while limiting interference with the surgeon's ability to access or see the working space. In some embodiments the blade assembly can be inserted over a screw by pressing the actuation end of the button 850 and sliding the blade assembly over the screw. In some embodiments, the blade assembly can be inserted without pressing the button. In some embodiments, pushing the blade assembly on the screw can move a locking device such as the button 850 into an unlocked position, allowing for the blade assembly to be moved down over the screw. In some embodiments where notches 752 (FIG. 29B) are of different sizes, the locking device can remain in an unlocked position until the blade assembly 830 is fully inserted, at which point the locking device can reach a notch that fits the locking device, which can move into a locked position within the notch. In some embodiments, as described above, when the blade assembly is fully inserted a portion of the central section 834 can be positioned against a distal end of the base portion 766 of a screw.

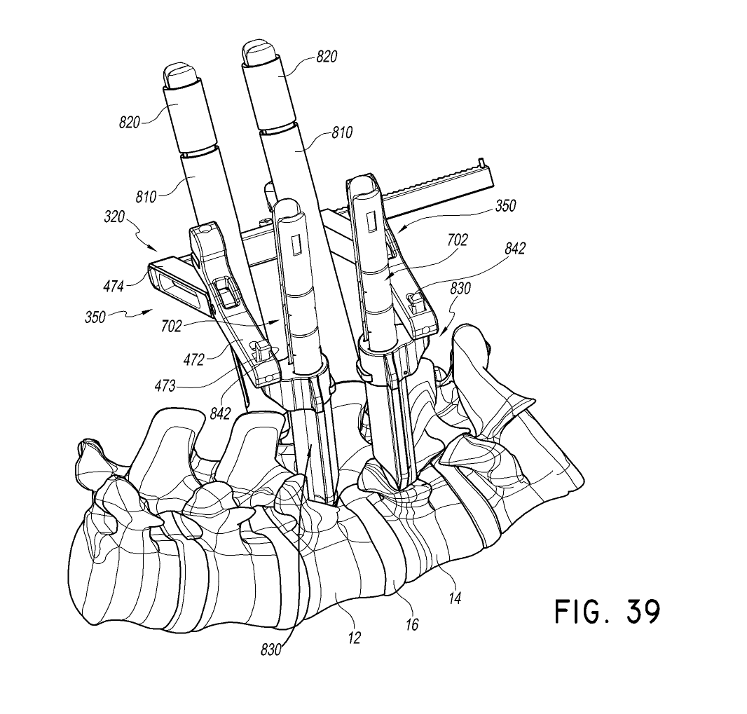

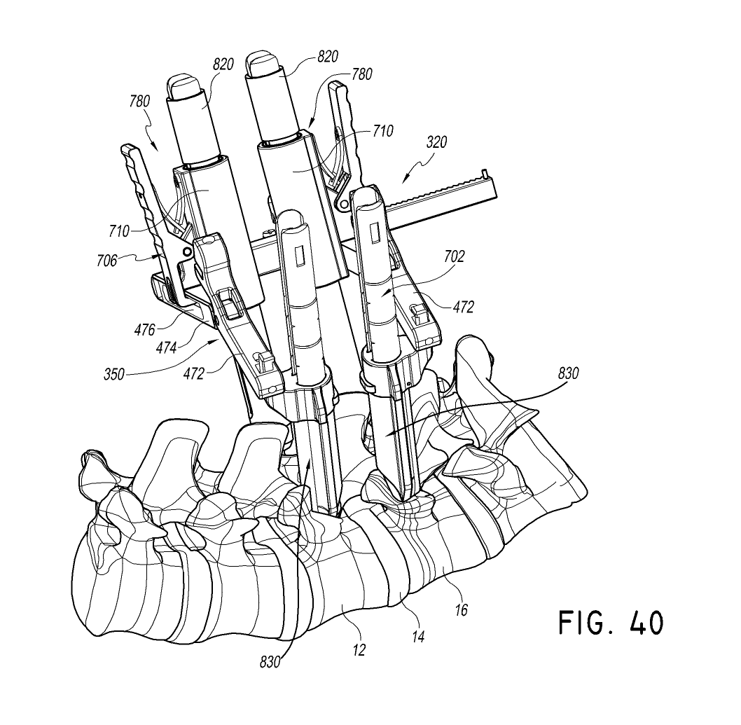

With reference to FIG. 39, in some embodiments a retractor body 320 can be placed into position relative to the screws 702 and/or any instrumentation inserted over the screws. Preferably, arms 350 of the retractor body 320 are positioned outside of the screws 702, such as illustrated, although in some embodiments one or more of the arms can be positioned between the screws. The arms can be attached or coupled to the screws 702 through a variety of means. In some embodiments, the arms can attach directly to the screws. In some embodiments, the arms can attach to instrumentation positioned around the screws.

For example, the posts 840 of blade assemblies 830 can be inserted through openings 473 in the arms. In some embodiments, the openings 473 can have approximately the same cross-sectional size and shape as a cross section of the posts 840 at projections 842 (shown in more detail in FIG. 30B). The portion of the post below the projection can have a smaller cross-sectional area, allowing for some movement of the post within the opening 473. When the post moves, the projection 842 can extend past the edge of the opening 473, helping prevent accidental removal of the post from the opening. In some embodiments, the projection 842 can be on a side of the post that faces a working area, such as the disc space or disc 16. This can tend to put the projection in a position to prevent accidental removal when the retractor body 320 is used to distract the disc space.

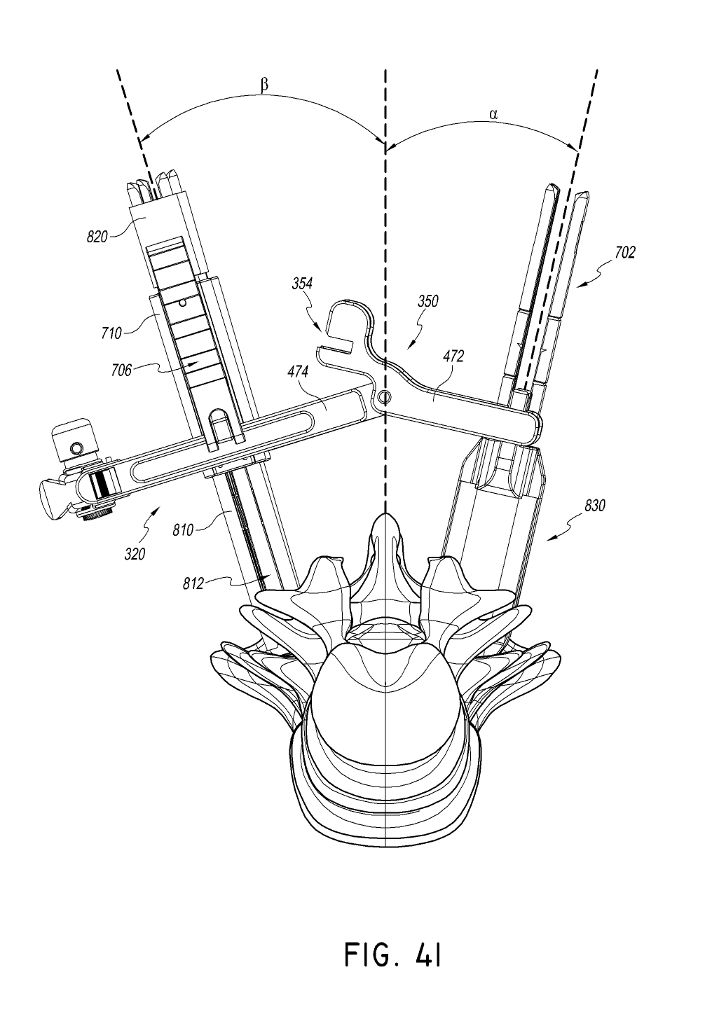

In some embodiments, the arms 350 can attach directly to a sleeve, such as the sleeve 810. In some embodiments, for example as shown in FIG. 40, a tube assembly 780 can be positioned over the sleeve 810 and/or the screw 702 and can attach to an arm 350, such as described above. FIG. 40 also illustrates one embodiment of a step in the procedure when both the operational and contralateral sides are attached to the retractor body 320. The retractor body can then be used to separate the arms 350, thereby distracting the disc space and retracting additional tissue. In some embodiments, either before or after distracting the disc space, a medial blade assembly 500, for example as shown in FIG. 28, can be attached and used to medially retract tissue. In some embodiments, a surgeon may improve access to the intervertebral space, such as described above, before or after distracting the disc space. Various procedures can be performed in the disc space.

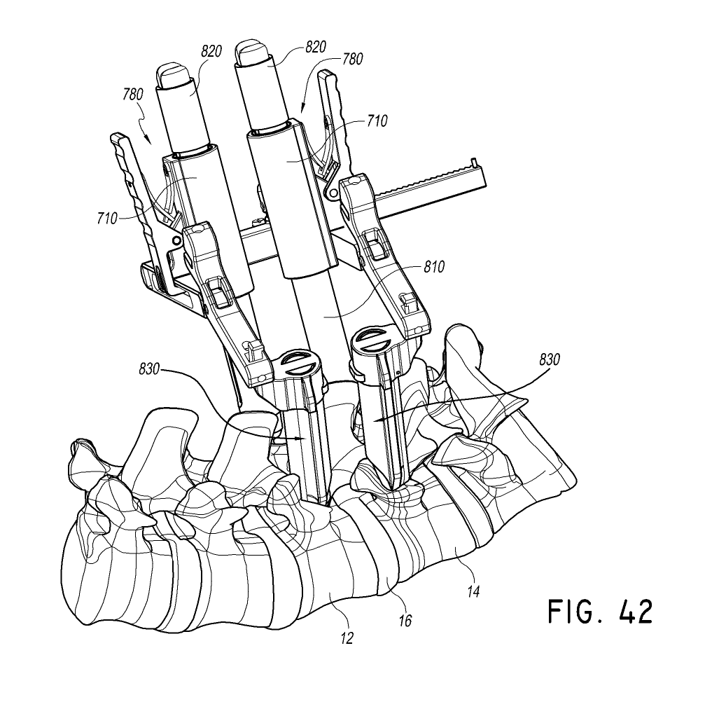

FIG. 41 illustrates a view in a transverse plane with the retractor body 320 in position. As illustrated, in some embodiments the screws 702 on the operational side can form an angle α with a spinal midline. In some embodiments, the screws on the contralateral side can form an angle β with the spinal midline. In some embodiments, the angle α and the angle β can be approximately equal. In some embodiments, the angle β can be greater than or less than to the angle α.

In some embodiments, before or after operating the retractor body 320 to distract the disc space, a portion of the screws 702 extending past the blades 830 on the operational side can be removed. This can lower the height of the screw and help limit any interference in access to the spinal disc space from screws extending past the blades. FIG. 42 illustrates an embodiment in which the screws have been broken at approximately the level of the top of the blade assemblies 830, such as where the tabs of the screws exited the blade assemblies. The screws can be broken, for example, at scored or otherwise frangible sections. In some embodiments this can be done manually. In some embodiments a tool can be inserted over a tab of the screw and angled to break the tab.

After the disc space has been distracted using the retractor, an optional procedure to improve access to the intervertebral space (e.g., a facetectomy) may be performed. An interbody cage may then be inserted into the disc space through the ipsilateral side. Once the desired procedure has been completed, the retractor body and any instrumentation inserted over the screws can be removed, for example as shown in FIG. 43. Once the instrumentation has been removed from the operational and/or contralateral sides, fusion rods can be inserted on that side. Rods can be positioned in the channels 772 in the screws and moved into position in the base portion 766 (FIG. 29B). In some embodiments, as described above, the sleeve 810 can be used to move the rods into position. In some embodiments, the rods can be moved manually into position. In some embodiments, for example where the screws are inserted percutaneously on the contralateral side, percutaneous instrumentation can be used to insert the fusion rods. Set screws delivered through the channel between the tabs can be used to lock the rods. Once a rod is in place, the remaining tabs 778 can be separated from the base portion of the screws 702 and the incisions can be closed.

Although the foregoing description of the preferred embodiments has shown, described and pointed out the fundamental novel features of the invention, it will be understood that various omissions, substitutions, and changes in the form of the detail of the apparatus as illustrated as well as the uses thereof, may be made by those skilled in the art, without departing from the spirit of the invention.

Reference throughout this specification to “one embodiment” or “an embodiment” means that a particular feature, structure or characteristic described in connection with the embodiment is included in at least one embodiment. Thus, appearances of the phrases “in one embodiment” or “in an embodiment” in various places throughout this specification are not necessarily all referring to the same embodiment. Furthermore, the particular features, structures or characteristics of any embodiment described above may be combined in any suitable manner, as would be apparent to one of ordinary skill in the art from this disclosure, in one or more embodiments.

Similarly, it should be appreciated that in the above description of embodiments, various features of the inventions are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that any claim require more features than are expressly recited in that claim. Rather, as the following claims reflect, inventive aspects lie in a combination of fewer than all features of any single foregoing disclosed embodiment. Thus, the claims following the Detailed Description are hereby expressly incorporated into this Detailed Description, with each claim standing on its own as a separate embodiment.

What is claimed is:

1. A minimally invasive surgical method for treating the spine, the method comprising:
delivering a first bone anchor into a first vertebra and a second bone anchor into a second vertebra, wherein the first bone anchor comprises a first shank and a first housing having a first extended tab and a first height, and wherein the second bone anchor comprises a second shank and a second housing having a second extended tab and a second height;
removing a first portion of the first extended tab such that the first housing has a third height less than the first height;
removing a first portion of the second extended tab such that the second housing has a fourth height less than the second height;
removing a second portion of the first extended tab after removing the first portion of the first extended tab, such that the first housing has a fifth height less than the third height; and
removing a second portion of the second extended tab after removing the first portion of the second extended tab, such that the second housing has a sixth height less than the fourth height.

2. The method of claim 1, wherein removing a first portion of the first extended tab comprises breaking the first portion off of the first extended tab.

3. The method of claim 1, wherein the third height is equal to the fourth height.

4. The method of claim 1, wherein the fifth height is equal to the sixth height.

5. The method of claim 1, further comprising positioning an attachment assembly over the first extended tab before removing the first portion of the first extended tab.

6. The method of claim 5, wherein the attachment assembly is a blade assembly.

7. The method of claim 1, wherein removing a second portion of the first extended tab comprises breaking the second portion off of the first extended tab.

8. The method of claim 1, further comprising inserting a surgical device into a working space before removing the second portion of the first extended tab and the second portion of the second extended tab, wherein at least a portion of the working space is between the first extended tab and the second extended tab.

9. The method of claim 8, wherein the surgical device is an implant.

10. The method of claim 8, wherein the surgical device is a tool.

11. The method of claim 1, wherein the first housing and/or the second housing is monolithic.

12. A minimally invasive surgical system for treating the spine, comprising:
at least one bone anchor comprising a shank and a housing having an extended tab, the housing having a first height from a distal tip of the housing to a proximal tip of the housing, the extended tab comprising a plurality of frangible sections;
an attachment assembly comprising an internal longitudinal bore sized to fit over at least a portion of the extended tab of the at least one bone anchor; and
a retractor assembly comprising a retractor arm configured to attach to the attachment assembly.

13. The minimally invasive surgical system of claim 12, wherein the attachment assembly has a second height from a distal tip of the attachment assembly to a proximal facing surface of the attachment assembly, the second height being less than the first height.

14. The minimally invasive surgical system of claim 12, wherein the retractor arm attaches directly to the attachment assembly.

15. The minimally invasive surgical system of claim 12, wherein the extended tab comprises two prongs defining a transverse channel between them.

16. The minimally invasive surgical system of claim 15, wherein the attachment assembly comprises an inward projection configured to fit within at least a portion of the transverse channel.

17. The minimally invasive surgical system of claim 12, further comprising a sleeve configured to be placed around a portion of the extended tab of at least one of the at least one bone anchors.

18. The minimally invasive surgical system of claim 17, wherein the sleeve is configured for placement between the extended tab of the at least one of the at least one bone anchors and an attachment assembly.

19. The minimally invasive surgical system of claim 12, wherein the attachment assembly comprises a lock that has a locked position and an unlocked position, and the lock in the locked position prevents longitudinal movement of the attachment assembly relative to the extended tab.

20. An extended tab pedicle screw, the pedicle screw comprising:
a screw shank; and
a housing attached to the screw shank, the housing comprising a plurality of frangible sections at different heights along the housing and two opposed side walls that define a transverse channel between them, the housing having a distal end nearest the screw shank and an opposite proximal end.

21. The extended tab pedicle screw of claim 20, wherein the screw shank is cannulated.

22. The extended tab pedicle screw of claim 20, wherein the plurality of frangible sections are separated by scores on the side walls.

23. The extended tab pedicle screw of claim 20, wherein the housing further comprises a plurality of locking notches positioned longitudinally along the housing.

24. The extended tab pedicle screw of claim 23, wherein at least two of the locking notches are different sizes.

25. The extended tab pedicle screw of claim 23, wherein a first locking notch is distal to and wider than a second locking notch.

26. The extended tab pedicle screw of claim 25, wherein each locking notch is wider than a more proximal locking notch or narrower than a more distal locking notch.

27. The extended tab pedicle screw of claim 23, wherein the plurality of locking notches are identical.

28. A minimally invasive surgical method for treating the spine of a patient, the method comprising:
inserting a first screw into a first vertebra and a second screw into a second vertebra on an ipsilateral side of the patient, the first and second screws each comprising a shaft, a base portion and an extended tab portion comprising two tabs and a channel between the tabs, the extended tab portions extending above a skin level of the patient after the first and second screws are inserted;
inserting a third screw into the first vertebra and a fourth screw into the second vertebra on a contralateral side of the patient, the third and fourth screws each comprising a shaft, a base portion and an extended tab portion comprising two tabs and a channel between the tabs, the extended tab portions extending above a skin level of the patient after the third and fourth screws are inserted;
positioning blade assemblies over the first and second screws on the ipsilateral side, wherein the extended tab portions of the first and second screws extend above the upper surfaces of the blade assemblies;
positioning one or more sleeves over the third and fourth screws on the contralateral side;
attaching a retractor assembly to the blade assemblies and the one or more sleeves;
removing upper portions of the extended tab portions of the first and second screws with remaining tab portions of the first and second screws positioned within the blade assemblies;
distracting a disc space between the first and second vertebrae using the retractor assembly;
inserting an interbody cage into the disc space on the ipsilateral side of the patient;
removing the retractor assembly from the blade assemblies and sleeves;
after removing the blade assemblies from the first and second screws, inserting a first rod into the base portions of the first and second screws;
after removing the sleeves from the third and fourth screws, inserting a second rod into the base portions of the third and fourth screws;
removing the remaining tab portions of the first and second screws from the base portions of the first and second screws; and
removing the extended tab portions of the third and fourth screws from the base portions of the third and fourth screws.

29. The method of claim 28, wherein the first and second screws each comprises a plurality of frangible or weakened sections, and wherein removing the upper portions of the extended tab portions of the first and second screws and removing the remaining tab portions of the first and second screws from the base portions of the first and second screws causes separation along the frangible or weakened sections.

30. The method of claim 28, wherein the third and fourth screws each comprises a frangible or weakened section between the extended tab portion and the base portion, and removing the extended tab portions of the third and fourth screws from the base portions of the third and fourth screws causes separation along the frangible or weakened section.

31. The method of claim 28, wherein the retractor assembly attaches to the blade assemblies through openings in arms of the retractor assembly.

32. The method of claim 28, wherein the retractor assembly attaches to the one or more sleeves with tubes attached to arms of the retractor assembly.

33. The method of claim 28, wherein removing the upper portions of the extended tab portions of the first and second screws occurs before distracting the disc space.

34. The method of claim 28, further comprising delivering set screws through the channels between the tabs of the screws, the set screws engaging threads in the base portions of the screws to fix the rods in place.

35. The method of claim 28, further comprising positioning a medial blade between the blade assemblies of the first screw and the second screw.

36. A minimally invasive surgical system for treating the spine, comprising:
first and second bone anchors each comprising a threaded shaft, a base portion configured to receive a rod, and an extended tab portion comprising two tabs and a channel between the tabs, wherein the first and second bone anchors each further comprises a frangible or weakened section between the base portion and the extended tab portion, and the extended tab portion comprising a plurality of additional frangible of weakened sections;
third and fourth bone anchors each comprising a threaded shaft, a base portion configured to receive a rod, and an extended tab portion comprising two tabs and a channel between the tabs, wherein the third and fourth bone anchors each further comprises a frangible or weakened section between the base portion and the extended tab portion;
first and second blade assemblies each having a central bore configured to slide over the extended tab portions of the first and second bone anchors, respectively, wherein the first and second blade assemblies have a length that is shorter than a length of the extended tab portions;
first and second sleeves each having a central bore configured to slide over the extended tab portions of the third and fourth bone anchors, respectively; and
a retractor assembly comprising a first arm and a second arm extending from a bar, wherein at least one of the arms is configured to move relative to the bar to change a distance between the arms;
wherein the first blade assembly and the first sleeve are configured to be attached to the first arm and the second blade assembly and the second sleeve are configured to be attached to the second arm.

37. The system of claim 36, wherein the arms each comprises a first section and a second section that can articulate relative to each other.

38. The system of claim 36, wherein each of the arms comprises a collar configured to fit over a corresponding post of the first and second blade assemblies.

39. The system of claim 36, further comprising first and second tube assemblies attached to the first and second arms, respectively, wherein each tube assembly comprises a tube configured to slide over one of the first and second sleeves.

40. The system of claim 36, wherein the first and second blade assemblies each further comprises an interior projection configured to fit between opposing tabs of the extended tab portions of the first and second bone anchors.

41. The system of claim 36, wherein the first and second blade assemblies each further comprises wing or blade extensions extending from a central section of each blade assembly.

42. The system of claim 36, wherein the first and second blade assemblies each further comprises a locking surface configured to engage notches on the first and second bone anchors.

43. The system of claim 36, further comprising a medial blade assembly attached to one or both arms, the medial blade assembly comprising a medial blade configured to be positioned between the first and second blade assemblies.

44. A minimally invasive surgical system for treating the spine, comprising:
a retractor assembly comprising a first arm and a second arm extending from a bar, wherein at least one of the arms is configured to move relative to the bar to change a distance between the arms;
first and second blade assemblies configured to engage a first bone anchor inserted into a first vertebra and a second bone anchor inserted into a second vertebra, respectively, on an ipsilateral side of a patient, wherein the first blade assembly is configured to be attached to the first arm and the second blade assembly is configured to be attached to the second arm; and
an attachment assembly attached to the first and second arms and configured to connect to extensions extending from third and fourth bone anchors inserted into the first and second vertebrae, respectively, on a contralateral side of the patient;
wherein the blade assemblies are configured to engage the first and second bone anchors by sliding over extended tab portions of the first and second bone anchors.

45. The system of claim 44, further comprising first and second bone anchors each comprising a threaded shaft, a base portion configured to receive a rod, and an extended tab portion comprising two tabs and a channel between the tabs.

46. The system of claim 45, wherein the first and second bone anchors each further comprises a frangible or weakened section between the base portion and the extended tab portion, and the extended tab portion comprising a plurality of additional frangible of weakened sections.

47. The system of claim 45, wherein the first and second blade assemblies each further comprises an interior projection configured to fit between opposing tabs of the extended tab portions of the first and second bone anchors.

48. The system of claim 44, wherein the attachment assembly comprises tubes configured to slide over the extensions extending from the third and fourth bone anchors.

49. The system of claim 48, further comprising third and fourth bone anchors each comprising a threaded shaft, a base portion configured to receive a rod, and an extended tab portion comprising two tabs and a channel between the tabs.

50. The system of claim 49, wherein the third and fourth bone anchors each further comprises a frangible or weakened section between the base portion and the extended tab portion.

51. The system of claim 44, wherein the arms each comprises a first section and a second section that can articulate relative to each other.

52. The system of claim 44, wherein each of the arms comprises a collar configured to fit over a corresponding post of the first and second blade assemblies.

53. The system of claim 44, wherein the first and second blade assemblies each further comprises wing or blade extensions extending from a central section of each blade assembly.

54. The system of claim 44, wherein the first and second blade assemblies each further comprises a locking surface configured to engage notches on the first and second bone anchors.

55. The system of claim 44, further comprising a medial blade assembly attached to one or both arms, the medial blade assembly comprising a medial blade configured to be positioned between the first and second blade assemblies.
